SEQUENCE LISTING (17 sequences)

SEQ ID NO: 1 - DNA, 21 bp, Artificial Sequence
  Description of Combined DNA/RNA Molecule Synthetic siRNA
  Description of Artificial Sequence Synthetic siRNA
cgggacucuagcauacuuatt

SEQ ID NO: 2 - DNA, 21 bp, Artificial Sequence
  Description of Combined DNA/RNA Molecule Synthetic siRNA
  Description of Artificial Sequence Synthetic siRNA
uaaguaugcuagagucccgtt

SEQ ID NO: 3 - DNA, 21 bp, Artificial Sequence
  Description of Combined DNA/RNA Molecule Synthetic siRNA
  Description of Artificial Sequence Synthetic siRNA
acugacagccagacagcgatt

SEQ ID NO: 4 - DNA, 21 bp, Artificial Sequence
  Description of Combined DNA/RNA Molecule Synthetic siRNA
  Description of Artificial Sequence Synthetic siRNA
ucgcugucuggcugucagutt

SEQ ID NO: 5 - DNA, 21 bp, Artificial Sequence
  Description of Combined DNA/RNA Molecule Synthetic siRNA
  Description of Artificial Sequence Synthetic siRNA
agacagcgaccaaaagaautt

SEQ ID NO: 6 - DNA, 21 bp, Artificial Sequence
  Description of Combined DNA/RNA Molecule Synthetic siRNA
  Description of Artificial Sequence Synthetic siRNA
auucuuuuggucgcugucutt

SEQ ID NO: 7 - DNA, 21 bp, Artificial Sequence
  Description of Combined DNA/RNA Molecule Synthetic siRNA
  Description of Artificial Sequence Synthetic siRNA
augaagaucuguuccaccatt

SEQ ID NO: 8 - DNA, 21 bp, Artificial Sequence
  Description of Combined DNA/RNA Molecule Synthetic siRNA
  Description of Artificial Sequence Synthetic siRNA
ugguggaacagaucuucautt

SEQ ID NO: 9 - DNA, 21 bp, Artificial Sequence
  Description of Combined DNA/RNA Molecule Synthetic siRNA
  Description of Artificial Sequence Synthetic siRNA
gaucuguuccaccauugaatt

SEQ ID NO: 10 - DNA, 21 bp, Artificial Sequence
  Description of Combined DNA/RNA Molecule Synthetic siRNA
  Description of Artificial Sequence Synthetic siRNA
uucaaugguggaacagauctt

SEQ ID NO: 11 - DNA, 21 bp, Artificial Sequence
  Description of Combined DNA/RNA Molecule Synthetic siRNA
  Description of Artificial Sequence Synthetic siRNA
gcaauugaggagugccugatt

SEQ ID NO: 12 - DNA, 21 bp, Artificial Sequence
  Description of Combined DNA/RNA Molecule Synthetic siRNA
  Description of Artificial Sequence Synthetic siRNA
ucaggcacuccucaauugctt

SEQ ID NO: 13 - DNA, 21 bp, Artificial Sequence
  Description of Combined DNA/RNA Molecule Synthetic siRNA
  Description of Artificial Sequence Synthetic siRNA
uugaggagugccugauuaatt

SEQ ID NO: 14 - DNA, 21 bp, Artificial Sequence
  Description of Combined DNA/RNA Molecule Synthetic siRNA
  Description of Artificial Sequence Synthetic siRNA
uuaaucaggcacuccucaatt

SEQ ID NO: 15 - DNA, 21 bp, Artificial Sequence
  Description of Combined DNA/RNA Molecule Synthetic siRNA
  Description of Artificial Sequence Synthetic siRNA
ggaucuuauuucuucggagtt

SEQ ID NO: 16 - DNA, 21 bp, Artificial Sequence
  Description of Combined DNA/RNA Molecule Synthetic siRNA
  Description of Artificial Sequence Synthetic siRNA
cuccgaagaaauaagaucctt

SEQ ID NO: 17 - DNA, 1565 bp, Influenza A virus
agcaaaagcagggtagataatcactcactgagtgacatcaaaatcatggcgtcccaaggcaccaaacggtcttacgaacagatggagactgatggagaacgccagaatgccactgaaatcagagcatccgtcggaaaaatgattggtggaattggacgattctacatccaaatgtgcaccgaactcaaactcagtgattatgagggacggttgatccaaaacagcttaacaatagagagaatggtgctctctgcttttgacgaaaggagaaataaatacctggaagaacatcccagtgcggggaaagatcctaagaaaactggaggacctatatacaggagagtaaacggaaagtggatgagagaactcatcctttatgacaaagaagaaataaggcgaatctggcgccaagctaataatggtgacgatgcaacggctggtctgactcacatgatgatctggcattccaatttgaatgatgcaacttatcagaggacaagagctcttgttcgcaccggaatggatcccaggatgtgctctctgatgcaaggttcaactctccctaggaggtctggagccgcaggtgctgcagtcaaaggagttggaacaatggtgatggaattggtcaggatgatcaaacgtgggatcaatgatcggaacttctggaggggtgagaatggacgaaaaacaagaattgcttatgaaagaatgtgcaacattctcaaagggaaatttcaaactgctgcacaaaaagcaatgatggatcaagtgagagagagccggaacccagggaatgctgagttcgaagatctcacttttctagcacggtctgcactcatattgagagggtcggttgctcacaagtcctgcctgcctgcctgtgtgtatggacctgccgtagccagtgggtacgactttgaaagagagggatactctctagtcggaatagaccctttcagactgcttcaaaacagccaagtgtacagcctaatcagaccaaatgagaatccagcacacaagagtcaactggtgtggatggcatgccattctgccgcatttgaagatctaagagtattaagcttcatcaaagggacgaaggtgctcccaagagggaagctttccactagaggagttcaaattgcttccaatgaaaatatggagactatggaatcaagtacacttgaactgagaagcaggtactgggccataaggaccagaagtggaggaaacaccaatcaacagagggcatctgcgggccaaatcagcatacaacctacgttctcagtacagagaaatctcccttttgacagaacaaccattatggcagcattcaatgggaatacagagggaagaacatctgacatgaggaccgaaatcataaggatgatggaaagtgcaagaccagaagatgtgtctttccaggggcggggagtcttcgagctctcggacgaaaaggcagcgagcccgatcgtgccttcctttgacatgagtaatgaaggatcttatttcttcggagacaatgcagaggagtacgacaattaaagaaaaatacccttgtttctact

(19) United States
(12) Patent Application Publication
(10) Pub. No.: US 2007/0172430 A1
(43) Pub. Date: Jul. 26, 2007

(54) DRY POWDER COMPOSITIONS FOR RNA INFLUENZA THERAPEUTICS

(75) Inventors: Luis Brito, Winchester, MA (US); Donghao Chen, Lexington, MA (US); Qing Ge, Rolla, MO (US); Doug Treco, Arlington, MA (US)

Correspondence Address: NASTECH PHARMACEUTICAL COMPANY INC, 3830 MONTE VILLA PARKWAY, BOTHELL, WA, 98021-7266, US

(73) Assignee: Nastech Pharmaceutical Company Inc.

(21) Appl. No.: 11/623,306

(22) Filed: Jan. 15, 2007

Related U.S. Application Data
(60) Provisional application No. 60/760,714, filed on Jan. 20, 2006.

(51) Int. Cl. A61K 48/00; A61K 9/14

(52) U.S. Cl. 424/46; 514/44

(57) ABSTRACT

A dry powder formulation for mucosal, intranasal, inhalation or pulmonary delivery which may include one or more siRNAs or dicer-active precursors thereof targeted to a transcript involved in infection by, or replication or production of an influenza virus.

This application claims the benefit under 35 U.S.C. § 119(e) of U.S. Provisional Application No. 60/760,714, filed Jan. 20, 2006, which is hereby incorporated by reference in its entirety.

### BACKGROUND OF THE INVENTION

In respiratory diseases such as influenza, the airway mucosal epithelium is a target of infection. Treatment for influenza should benefit from administration of antiviral or ameliorative agents directly to the airway epithelium. In addition, the risk of a serious influenza outbreak is significant. New therapies to treat various influenza viral infections are presently needed.

RNA Interference (RNAi) refers to methods of sequence-specific post-transcriptional gene silencing which is mediated by a double-stranded RNA (dsRNA) called a short interfering RNA (siRNA). See Fire, et al., Nature 391:806, 1998, and Hamilton, et al., Science 286:950-951, 1999. RNAi is shared by diverse flora and phyla and is believed to be an evolutionarily-conserved cellular defense mechanism against the expression of foreign genes. See Fire, et al., Trends Genet. 15:358, 1999.

RNAi is therefore a ubiquitous, endogenous mechanism that uses small noncoding RNAs to silence gene expression. See Dykxhoorn, D. M. and J. Lieberman, Annu. Rev. Biomed. Eng. 8:377-402, 2006. RNAi can regulate important genes involved in cell death, differentiation, and development. RNAi may also protect the genome from invading genetic elements, encoded by transposons and viruses. When a siRNA is introduced into a cell, it binds to the endogenous RNAi machinery to disrupt the expression of mRNA containing complementary sequences with high specificity. Any disease-causing gene and any cell type or tissue can potentially be targeted. This technique has been rapidly utilized for gene-function analysis and drug-target discovery and validation. Harnessing RNAi also holds great promise for therapy, although introducing siRNAs into cells in vivo remains an important obstacle.

The mechanism of RNAi, although not yet fully characterized, is through cleavage of a target mRNA. The RNAi response involves an endonuclease complex known as the RNA-induced silencing complex (RISC), which mediates cleavage of a single-stranded RNA complementary to the antisense strand of the siRNA duplex. Cleavage of the target RNA takes place in the middle of the region complementary to the antisense strand of the siRNA duplex (Elbashir, et al., Genes Dev. 15:188, 2001).

Mechanistically, it is now known that when one uses long dsRNA in organisms such as plants, the long dsRNA is first cleaved into short interfering RNAs (siRNAs, 19-25 bp duplexes) by Dicer, a Type III RNase. Subsequently, these small duplexes interact with the RNA Induced Silencing Complex (RISC), a multisubunit complex containing both helicases and endonuclease activities that mediate degradation of homologous transcripts.

It has been discovered that mammalian cells transfected with synthetic siRNAs could induce the RNAi pathway (Elbashir, S. M., et al., “Duplexes of 21-nucleotide RNAs mediate RNA interference in cultured mammalian cells, ” Nature 411(6836):494-8, 2001. The ability to target a wide range of gene transcripts with short interfering RNAs, and the specificity of siRNA-mediated gene knockdown suggests that this approach may be useful for therapeutic applications.

One way to carry out RNAi is to introduce or express a siRNA in cells. Another way is to make use of an endogenous ribonuclease III enzyme called dicer. One activity of dicer is to process a long dsRNA into siRNAs. See Hamilton, et al., Science 286:950-951, 1999; Berstein, et al., Nature 409:363, 2001. A siRNA derived from dicer is typically about 21-23 nucleotides in overall length with about 19 base pairs duplexed. See Hamilton, et al., supra; Elbashir, et al., Genes Dev. 15:188, 2001. In essence, a long dsRNA can be introduced in a cell as a precursor of a siRNA.

The development of RNAi has created a need for effective means of introducing active nucleic acid-based agents into cells. In general, nucleic acids are stable for only limited times in cells or plasma.

Therapeutic reagents such as a siRNA for treating a pulmonary disease may be delivered to diseased tissues by a variety of routes. However, oral and intraveneous-administration have drawbacks including side effects associated with indirect methods of delivery, patient aversion to needle-based delivery methods, and degradation of the active pharmaceutical ingredient in the bloodstream and gastric environment.

Direct pulmonary delivery is a route of administration having advantages over parenteral administration including convenience of patient self-administration, the potential for reduced drug side-effects, ease of delivery by inhalation, and the elimination of needles.

Preclinical and clinical studies with inhaled proteins, peptides, and small molecules have demonstrated that efficacy can be achieved both within the lungs and systemically as direct pulmonary delivery can result in relatively high bioavailability of many molecules, including macromolecules, Wall, D. A., Drug Delivery 2:1-20, 1995; Patton, J. and R. Platz, Adv. Drug Del. Rev. 8:179-196, 1992; and Byron, P., Adv. Drug. Del. Rev. 5:107-132, 1990.

One methodology for delivering therapeutics to the lungs is dry powder formulation (DPF); Damms, B. and W. Bains, Nature Biotechnology, 1996; Kobayashi, S., et al., Pharm. Res. 13(1):80-83, 1996; and Timsina, M., et al., Int. J. Pharm. 101:1-13, 1994. DPF aerosols for inhalation therapy are applicable to a range of biomolecules and offer pulmonary distribution when formulated and delivered with desired chemical/physical properties and optimal dosing regimes; Ganderton, D., J. Biopharmaceutical Sciences 3:101-105, 1992; and Gonda, I., “Physico-Chemical Principles in Aerosol Delivery,” in Topics in Pharmaceutical Sciences, 1991; Crommelin, D. J. and K. K. Midha, eds., Medpharmn Scientific Publishers, Stuttgart, pp. 95-115, 1992. Large “carrier” particles (containing no drug) have been co-delivered with therapeutic aerosols to aid in achieving efficient aerosolization among other possible benefits. French, D. L., Edwards, D. A. and Niven, R. W., J. Aerosol Sci. 27:769-783, 1996.

Powders consisting of fine particulates may have poor flowability and aerosolization properties, leading to relatively low respirable fractions of aerosol, which are the fractions of inhaled aerosol that deposit in the lungs, escaping deposition in the mouth and throat. Gonda, I., in Topics in Pharmaceutical Sciences, 1991, D. Crommelin and K. Midha, Editors, Stuttgart: Medpharm Scientific Publishers, 95-117 (1992). Poor flowability and aerosolization properties are typically caused by particulate aggregation that results from hydrophobic, electrostatic, and capillary interactions. As these effects must be minimized in order to achieve effective inhalation therapies, various methods have been employed to prepare DPFs. These approaches include (1) the modification of dry powder particle surface texture (Ganderton, et al., U.S. Pat. No. 5,376,386), (2) the co-delivery of large carrier particles (absent drug) with therapeutic aerosols to achieve efficient aerosolization, particle coatings (Hanes, U.S. Pat. No. 5,855,913; Ruel, et al., U.S. Pat. No. 5,663,198), (3) the development of aerodynamically light particles (Edwards, et al., U.S. Pat. No. 5,985,309), (4) the use of antistatic agents, (Simpkin, et al., U.S. Pat. No. 5,908,639), and (5) the addition of certain excipients, e.g., surfactants (Hanes, U.S. Pat. No. 5,855,913; Edwards, U.S. Pat. No. 5,985,309).

What is needed are compositions and methods for administering active therapeutic agents such as for RNA interference to the lung and airways for pulmonary, pulmonary surface, and systemic effects. Suitable dry powder formulations are needed for pulmonary delivery of nucleic acids including small interfering RNAs (siRNAs). This includes formulations which avoid duplex denaturation during aerosolization, degradation of the active agent by nucleases, and excessive loss of the inhaled drug in the oropharyngeal cavity.

### BRIEF SUMMARY OF THE INVENTION

This invention overcomes these and other drawbacks in the field by providing a range of ribonucleic acid compositions for use in RNA Interference and other therapeutic methods. This invention particularly provides compositions and methods of making compositions comprising one or more ribonucleic acid agents in a dry powder formulation which are active to inhibit expression of targeted genes through RNA Interference.

This invention relates generally to the fields of RNA Interference, and delivery of RNA therapeutics. More particularly, this invention relates to dry powder compositions of an RNA active in RNA Interference, and their uses for medicaments and for delivery as therapeutics for influenza. This invention relates generally to methods of using an RNA active in RNA Interference for gene-specific inhibition of gene expression in mammals. The dry powder compositions of this invention may be used for aerosolized delivery to the lungs.

In some embodiments, this invention includes dry powder formulations for aerosolization and delivery to the lung which provide enhanced delivery of nucleic acids, such as siNAs.

In some embodiments, the dry powder particles of this invention have a mass median diameter of from about 0.7 to about 10.0 micrometers, or a mass median aerodynamic diameter of from about 1 to about 6 micrometers. In some embodiments, the dry powder includes particles having a density of from about 0.01 to about 2 grams per cubic centimeter. In some embodiments, the dry powder contains less than about 6% moisture. In some embodiments, at least about 90% of the particles are less than 8 micrometers in mass median diameter.

In some embodiments, the dry powder of this invention is characterized by both physical and chemical stability upon storage. In some embodiments, the chemical stability of the dry powder is characterized by degradation of less than about 10% by weight of the active RNA agent upon storage of the dry powdered composition under ambient conditions for a period of 18 months.

In other embodiments, this invention provides a method for manufacturing a DPF with an active agent such as a siNA. The process includes reconstituting the active agent in an aqueous solution optionally comprising of sugars, salts, peptides, proteins and/or polymers that are soluble in aqueous solutions such as PEG. Subsequently, the active agent in the aqueous phase is combined with an organic solution optionally containing lipids and polymers such as poly(lactide-co-glycolide) or PLGA that are soluble in organic mixtures. This mixture can be spray dried.

In some embodiments, spray drying is accomplished by expelling the mixture through a two fluid nozzle or other type of atomizer along with an inert gas maintained at temperatures ranging from 65-125 degrees Celsius. The gas and dry powder can then be separated, and particles having the desired physical, chemical, stability, and therapeutic properties collected. Alternatively, the active agent in an aqueous solution is precipitated from solution by adding salt and an organic solvent (e.g., ethanol). The organic solvent used to precipitate the active agent may also contain additional excipients (e.g., lipids, surfactants) that control the size and extent of precipitation of the active agent. Subsequently, the solution containing the precipitated active agent can be combined with an organic solution containing the desired non-water soluble excipients and spray dried. Additionally the active agent can be added to an aqueous solution containing various water soluble excipients. The aqueous solution can then be added to a non-miscible organic solution containing non-water soluble excipients. The two liquids are then homogenized. Additional water is added to the emulsion, to increase the amount of water in the water emulsion. This will encapsulate the active ingredient and other water soluble excipients within the non water soluble excipients after spray drying. As a result of these procedures, a DPF that contains the active agent and is capable of enhancing the therapeutic effect of the active agent over treatments that utilize naked (unformulated) active agent is formed.

In some embodiments, the active agent is a nucleic acid, particularly an oligonucleotide(s) that may be single or double stranded RNA. The oligonucleotide(s) may be a siRNA.

Another aspect of the invention is directed to a method for delivery of a dry powder composition to the lungs of a mammalian subject by administering by inhalation the compositions and formulations of this invention in aerosolized form.

A dry powder formulation for mucosal, intranasal, inhalation or pulmonary delivery may include one or more siRNAs or dicer-active precursors thereof targeted to a transcript involved in infection by, or replication or production of an influenza virus.

A dry powder aerosolizable formulation for mucosal, intranasal, inhalation or pulmonary delivery may include one or more siRNAs along with DPPC and a carrier such as lactose. The formulation may further include a buffer agent such as calcium chloride, a protein such as albumin, and an amino acid such as arginine.

In another aspect, this invention encompasses a method of treating or preventing influenza in an animal comprising administering a therapeutically effective amount of a dry powder formulation of a siNA to the animal.

In another aspect, this invention encompasses a method of inhibiting the replication or production of an influenza virus in an animal comprising administering a therapeutically effective amount of a dry powder formulation of a siNA to the animal.

These and other objects and features of the invention will become apparent when the following detailed description is read in conjunction with the accompanying figures and examples.

### BRIEF DESCRIPTION OF THE FIGURES

FIG. 1 depicts an example of a process for dry powder manufacturing.

FIG. 2 shows the in vivo reduction of PR8 influenza viral titer in Balb/c mice using dry powder formulations (lot 22-22, placebo; lot 22-23, active formulation of siRNA/DPPC/sucrose/albumin, 20/40/20/20; lot 22-14, placebo; and lot 22-16, active formulation of siRNA/DPPC/lactose/protamine, 20/45/30/5). Viral titer was characterized by tissue culture infectious dose (TCID₅₀) using a hemagglutination assay of chicken RBC. Each point represents a single animal.

FIG. 3 shows the in vivo reduction of PR8 influenza viral titer in Balb/c mice using dry powder formulations (lot 22-38, placebo; lot 22-42, active formulation of siRNA/DPPC/sucrose/arginine, 20/45/30/5). Viral titer was characterized by tissue culture infectious dose (TCID₅₀) using a hemagglutination assay of chicken RBC. Each point represents a single animal.

FIG. 4 shows the in vivo reduction of PR8 influenza viral titer in Balb/c mice using dry powder formulations (lot 22-18, placebo; lot 22-20, active formulation of siRNA/DPPC/lactose/calcium chloride, 20/47/30/3). Viral titer was characterized by tissue culture infectious dose (TCID₅₀) using a hemagglutination assay of chicken RBC. Each point represents a single animal.

FIG. 5 shows the in vivo reduction of PR8 influenza viral titer in Balb/c mice using dry powder formulations (lot 22-65, placebo; lot 22-67, active formulation of siRNA/DPPC/leucine/calcium chloride, 20/47/30/3). Viral titer was characterized by tissue culture infectious dose (TCID₅₀) using a hemagglutination assay of chicken RBC. Each point represents a single animal.

FIG. 6 shows the in vivo reduction of PR8 influenza viral titer in Balb/c mice using dry powder formulations (lot 22-18, placebo; lot 22-69, active formulation of siRNA/DPPC/lactose/calcium chloride, 20/47/30/3). Viral titer was characterized by tissue culture infectious dose (TCID₅₀) using a hemagglutination assay of chicken RBC. Each point represents a single animal.

FIG. 7 shows the in vivo reduction of PR8 influenza viral titer in Balb/c mice using dry powder formulations (lot 22-18, placebo; lot 22-73, active formulation of siRNA/DPPC/lactose/calcium chloride, 20/47/30/3). Viral titer was characterized by tissue culture infectious dose (TCID₅₀) using a hemagglutination assay of chicken RBC. Each point represents a single animal.

### DETAILED DESCRIPTION OF THE INVENTION

This invention encompasses delivery of RNA therapeutics, and more particularly, dry powder compositions of an RNA active in RNA Interference, and their uses for medicaments and for delivery as therapeutics for influenza. Methods and compositions of siNAs active for RNA Interference are provided for gene-specific inhibition of gene expression in mammals.

In some embodiments, this invention includes formulations of an siNA, including aerosol formulations and aerosolizable formulations. Dry powder compositions of this invention may be used for aerosolized delivery to the lungs.

Dry powder formulations of this invention can contain one or more carbohydrates, lipids, salts, peptides, proteins, and/or surfactants, and exhibit physical and chemical stability upon storage. Importantly, the dry powder formulations of this invention demonstrate superior aerosol performance for delivery of small interfering RNAs (siRNAs).

This invention addresses needs in the art and identifies compositions and manufacturing procedures that promote efficient pulmonary delivery of oligonucleotide(s). Such compositions and procedures enhance the effectiveness of nucleic acid delivery to the lung, thus enhancing the effectiveness of the active agent.

The dry powder formulations of this invention are effective for delivering agents to treat pulmonary diseases, and dry powder mediated delivery of drugs to the deep lung may also provide systemic delivery, and thus provide an efficient drug delivery methodology for treatment of systemic viral infections.

“Active agent” as described herein includes any substance that produces the response of RNAi Interference in a cell, whether in vivo or in vitro, such as a small interfering RNA.

As used herein, the terms “short interfering nucleic acid,” “siNA,” “short interfering RNA,” “siRNA,” “short interfering nucleic acid molecule,” “short interfering oligonucleotide molecule,” and “chemically-modified short interfering nucleic acid molecule,” refer to any nucleic acid molecule capable of inhibiting or down regulating gene expression or viral replication, for example, by mediating RNA interference (RNAi) or gene silencing in a sequence-specific manner.

“siNA” means a small interfering nucleic acid, for example a siRNA, that is a short-length double-stranded nucleic acid, or optionally a longer precursor thereof. The length of useful siNAs within this invention will in some embodiments be optimized at a length of approximately 20 to 50 bp. However, there is no particular limitation to the length of useful siNAs, including siRNAs. For example, siNAs can initially be presented to cells in a precursor form that is substantially different than a final or processed form of the siNA that will exist and exert gene silencing activity upon delivery, or after delivery, to the target cell. Precursor forms of siNAs may, for example, include precursor sequence elements that are processed, degraded, altered, or cleaved at or after the time of delivery to yield a siNA that is active within the cell to mediate gene silencing. In some embodiments, useful siNAs will have a precursor length, for example, of approximately 100-200 base pairs, or 50-100 base pairs, or less than about 50 base pairs, which will yield an active, processed siNA within the target cell. In other embodiments, a useful siNA or siNA precursor will be approximately 10 to 49 bp, or 15 to 35 bp, or about 21 to 30 bp in length.

“Aerosolized” or “aerosolizable” particles are particles which, when dispensed into a gas stream by either a passive or an active inhalation device, remain suspended in the gas for an amount of time sufficient for at least a portion of the particles to be inhaled by the subject so that a portion of the particles reaches the lungs. In this instance, the term “subject” includes any of a large number of animals including but not limited to mammals (such as humans and other primates, cows, pigs), birds (such as chickens, geese, and ducks), and reptiles.

“Amino acid” refers to any compound containing both an amino group and a carboxylic acid group. Although the amino group and the carboxylic acid group are most commonly attached to the same carbon atom (the “alpha” carbon), the amino group may be positioned at any location within the molecule. The amino acid may also contain additional functional groups, such as amino, thio, carboxyl, guanidinium, carboxamide, imidazole, etc. An amino acid may be synthetic or naturally occurring, and may be used in either its racemic or optically active (D- or L-) form.

“Atomization” or “atomized” refers to a process of separating and or inducing the article of the invention into fine droplets. Thus for instance, the process of manufacturing the dry powder of the invention, the formulation solution is atomized to create droplets that are subsequently dried having the proper size and aerodynamic properties for delivery to the pulmonary tissues.

The phrase “antisense region” refers to a sequence of nucleotides in a polynucleotide that is complementary to a sense region in the same polynucleotide (if the polynucleotide is a unimolecular polynucleotide having both a sense and antisense sequence, wherein the sense and antisense sequences are capable of annealing by reason of the polynucleotide forming intramolecular interactions such as, for example, a hairpin structure), or in a different polynucleotide (in the case of a double stranded polynucleotide that comprises two separate strands, one bearing a sense sequence and one bearing an antisense sequence, wherein the sense and antisense sequences are capable of annealing by reason of the two strands undergoing an intermolecular interaction to form, for example, a duplex).

The term “complementary” refers to the ability of polynucleotides to form base pairs with one another. Base pairs are typically formed by hydrogen bonds between nucleotide units in antiparallel polynucleotide strands. Complementary polynucleotide strands can base pair in the Watson-Crick manner (e.g., A to T, A to U, C to G), or in any other manner that allows for the formation of duplexes. As persons skilled in the art are aware, when using RNA as opposed to DNA, uracil rather than thymine is the base that is considered to be complementary to adenosine. However, when a U is denoted in the context of the present invention, the ability to substitute a T is implied, unless otherwise stated.

Complementarity refers to the situation in which each nucleotide unit of one polynucleotide strand can hydrogen bond with a nucleotide unit of a second polynucleotide strand. Complementarity may be perfect, less than perfect, or substantial. For example, two polynucleotides of 29 nucleotide units each, wherein each comprises a single-stranded or unpaired sequence of two deoxythymidine residues (di-dT or dTdT) at the 3′ terminus such that the duplex region spans 27 bases, and wherein 26 of the 27 bases of the duplex region on each strand are complementary, are substantially complementary since they are 96.3% complementary when excluding the di-dT overhangs.

“Delivery efficiency” as used herein refers to an experimentally determined value that provides an indication of the amount of powder delivered to an animal during an experiment. For example, an insuffulator containing a predetermined amount of powder is dosed to an animal. The weight of the insuffulator is taken before and after administration in addition to the predetermined weight of the powder. The DE is then calculated by subtracting the weight after administration from the weight before administration, divided by the predetermined weight of powder.

“Dipeptide,” also referred to herein as a dimer, refers to a peptide composed of two amino acids.

The phrase “duplex region” refers to the region in two complementary or substantially complementary polynucleotides that form base pairs with one another, either by Watson-Crick base pairing or any other manner that allows for a stabilized duplex between polynucleotide strands that are complementary or substantially complementary. For example, a polynucleotide strand having 21 nucleotide units can base pair with another polynucleotide of 21 nucleotide units, yet only 19 bases on each strand are complementary or substantially complementary, such that the “duplex region” has 19 base pairs. The remaining bases may, for example, exist as 5′ or 3′ overhangs. Further, within the duplex region, 100% complementarity is not required; substantial complementarity is allowable within a duplex region.

“Dry powder” refers to a powder composition that typically contains less than about 20% moisture, or less than 10% moisture, or less than about 6% moisture, or less than about 3% moisture. In this context, the term “moisture” is defined as the ratio of the mass of water present in the sample to the mass of the sample.

A dry powder that is “suitable for pulmonary delivery” refers to a composition comprising solid (i.e., non-liquid) or partially solid particles that are capable of being (i) readily dispersed in/by an inhalation device and/or (ii) inhaled by a subject so that a portion of the particles reach the lungs to permit penetration into the alveoli or other pulmonary anatomical structure. Such a powder is considered to be “respirable.”

“Emitted Dose” or “ED” provides an indication of the delivery of a drug formulation from a suitable inhaler device after a firing or dispersion event. More specifically, for dry powder formulations, the ED is a measure of the percentage of powder which is drawn out of a unit dose package and which exits the mouthpiece of an inhaler device. The ED is defined as the ratio of the dose delivered by an inhaler device to the nominal dose (i.e., the mass of powder per unit dose placed into a suitable inhaler device prior to firing). The ED is an experimentally determined parameter, and is typically determined using an in vitro device that mimics subject dosing. The DE of an insuffulator may differ from the ED of an inhaler.

“Fine particle fraction” or “FPF” is defined as the mass percent of powder particles having an aerodynamic diameter less than 5.6 μm, typically determined by measurement in an Andersen cascade impactor. This parameter provides an indication of the percent of particles having the greatest potential to reach the deep lung of a patient for systemic uptake of a drug substance.

A “dispersible” or “dispersive” powder is one having an ED value of at least about 30%, more preferably 40-50%, and even more preferably at least about 50-60%.

The phrase “gene silencing” refers to a process by which the expression of a specific gene product is lessened or attenuated. Gene silencing can take place by a variety of pathways. Unless specified otherwise, as used herein, gene silencing refers to decreases in gene product expression that results from RNA interference (RNAi).

The phrase “guide strand” is defined as the oligonucleotide strand of an siRNA that is designed to bind to the mRNA target in a RISC mediated manner. As used herein, the guide strand is synonymous with the antisense strand of the siRNA.

The phrase “internucleotide linkage” refers to the type of bond or linkage that is present between two nucleotide units in a polynucleotide and may be modified or unmodified. The phrase “internucleotide linkage modification” includes all modified internucleotide linkages now known in the art or that come to be known and that, from reading this disclosure, one skilled in the art will conclude is useful in connection with the present invention. Internucleotide linkages may have associated counterions, and the term is meant to include such counterions and any coordination complexes that can form at the internucleotide linkages.

Modifications of internucleotide linkages include, but are not limited to, phosphorothioates, phosphorodithioates, methylphosphonates, 5′-alkylenephosphonates, 5′-methylphosphonate, 3′-alkylene phosphonates, borontrifluoridates, borano phosphate esters and selenophosphates of 3′-5′ linkage or 2′-5′ linkage, phosphotriesters, thionoalkylphosphotriesters, hydrogen phosphonate linkages, alkyl phosphonates, alkylphosphonothioates, arylphosphonothioates, phosphoroselenoates, phosphorodiselenoates, phosphinates, phosphoramidates, 3′-alkylphosphoramidates, aminoalkylphosphoramidates, thionophosphoramidates, phosphoropiperazidates, phosphoroanilothioates, phosphoroanilidates, ketones, sulfones, sulfonamides, thioesters, carbonates, carbamates, methylenehydrazos, methylenedimethylhydrazos, formacetals, thioformacetals, oximes, methyleneiminos, methylenemethyliminos, thioamidates, linkages with riboacetyl groups, aminoethyl glycine, silyl or siloxane linkages, alkyl or cycloalkyl linkages with or without heteroatoms of, for example, 1 to 10 carbons that can be saturated or unsaturated and/or substituted and/or contain heteroatoms, linkages with morpholino structures, amides, polyamides wherein the bases can be attached to the aza nitrogens of the backbone directly or indirectly, and combinations of such modified internucleotide linkages within a polynucleotide.

“Mass median aerodynamic diameter” or “MMAD” is a measure of the aerodynamic size of a dispersed particle. The aerodynamic diameter is used to describe an aerosolized powder in terms of its settling behavior, and is the diameter of a unit density sphere having the same settling velocity, in air, as the particle. The aerodynamic diameter encompasses particle shape, density and physical size. As used herein, MMAD refers to the midpoint or median of the aerodynamic particle size distribution of an aerosolized powder determined by cascade impaction, unless otherwise indicated.

“Mass median diameter” or “MMD” is a measure of mean particle size, since the powders of the invention are generally polydisperse (i.e., consist of a range of particle sizes). MMD values as reported herein are determined by centrifugal sedimentation, although any number of commonly employed techniques can be used for measuring mean particle size (e.g., electron microscopy, light scattering, laser diffraction).

The term “mismatch” refers to instances in which non-classical (e.g., A-C, A-G, A-A, G-G, etc.) base pairing exists, but excludes “wobble” base-pairing (e.g., G-U).

The term “nucleotide” refers to a ribonucleotide or a deoxyribonucleotide or modified form thereof, as well as an analog thereof. Nucleotides include species that comprise purines, e.g., adenine, hypoxanthine, guanine, and their derivatives and analogs, as well as pyrimidines, e.g., cytosine, uracil, thymine, and their derivatives and analogs.

Nucleotide analogs include nucleotides having modifications in the chemical structure of the base, sugar and/or phosphate, including, but not limited to, C5 pyrimidine modifications (such as 5-propynyl uridine), C8 purine modifications, modifications at cytosine exocyclic amines, and substitution of 5-bromo-uracil; and 2′-position sugar modifications, including but not limited to, sugar-modified ribonucleotides in which the 2′-OH is replaced by a group such as an H, OR, R, halo, SH, SR, NH₂, NHR, NR₂, or CN, wherein R is an alkyl moiety as defined herein. Nucleotide analogs are also meant to include nucleotides with bases such as diaminopurine, inosine, queuosine, xanthine, sugars such as 2′-methyl ribose, threose, and glycerol, non-natural phosphodiester linkages such as methylphosphonates, phosphorothioates and peptide nuclei acids.

The phrases “off-target silencing” and “off-target interference” are defined as gene silencing of mRNA other than the intended target mRNA. Gene silencing due to off-targeting is RNAi dependent, results in transcript degradation or translation inhibition, and is due to overlapping and/or partial homology between the sense or antisense strand of the siRNA and the unintended target mRNA.

The term “overhang” refers to terminal non-base pairing nucleotide(s) resulting from one strand extending beyond the terminus of the complementary strand to which the first strand forms a doubled stranded polynucleotide. One or both of two polynucleotides that are capable of forming a duplex through hydrogen bonding of base pairs may have a 5′ and/or 3′ end that extends beyond the 3′ and/or 5′ end of complementarity shared by the two polynucleotides. The single-stranded region extending beyond the 3′ and/or 5′ end of the duplex is referred to as an overhang.

“Pharmaceutically acceptable salt” includes, but is not limited to, salts prepared with inorganic acids, such as chloride, sulfate, phosphate, diphosphate, hydrobromide, and nitrate salts, or salts prepared with an organic acid, such as malate, maleate, fumarate, tartrate, succinate, ethylsuccinate, citrate, acetate, lactate, methanesulfonate, benzoate, ascorbate, para-toluenesulfonate, palmoate, salicylate and stearate, as well as estolate, gluceptate and lactobionate salts. Similarly, salts containing pharmaceutically acceptable cations include, but are not limited to, sodium, potassium, calcium, aluminum, lithium, and ammonium (including alkyl substituted ammonium).

“Pharmaceutically acceptable excipient or carrier” refers to an excipient that may optionally be included in the compositions of the invention, and taken into the lungs with no significant adverse toxicological effects to the subject, and particularly to the lungs of the subject.

“Pharmacologically effective amount” or “physiologically effective amount of a bioactive agent” is the amount of an active agent present in an aerosolizable composition as described herein that is needed to provide a desired level of active agent in the bloodstream or at the site of action (e.g., the lung tissue) of a subject to be treated to give an anticipated physiological response when such composition is administered by pulmonary administration. The precise amount will depend upon numerous factors, e.g., the active agent, the activity of the active agent, the delivery device employed, the physical characteristics of the active agent, intended use by the subject (i.e., the number of doses administered per day), subject considerations, and the like, and can readily be determined by one skilled in the art, based upon the information provided herein.

“Polymer” refers to a high molecular weight compound or macromolecule consisting of a long chain of monomers linked to form a series of repeating units. A polymer may be a biological polymer, i.e., is naturally occurring (e.g., proteins, carbohydrates, nucleic acids) or a non-biological, synthetically-produced polymer (e.g., polyethylene glycols, polyvinylpyrrolidones, Ficolls, and the like) known in the art and may be comprised of identical or different chemical units.

In a polynucleotide or oligonucleotide, phosphate groups covalently link adjacent nucleosides to form a polymer. The polymer may comprise of natural nucleosides found in DNA or RNA (e.g., adenosine, thymidine, guanosine, cytidine, uridine, deoxyadenosine, deoxythymidine, deoxyguanosine, and deoxycytidine), other nucleosides or nucleoside analogs, nucleosides containing chemically modified bases and/or biologically modified bases (e.g., methylated bases), intercalated bases, modified sugars, etc. The phosphate groups in a polynucleotide or oligonucleotide are typically considered to form the internucleoside backbone of the polymer. In naturally occurring nucleic acids (DNA or RNA), the backbone linkage is via a 3′ to 5′ phosphodiester bond. However, polynucleotides and oligonucletides containing modified backbones or non-naturally occurring internucleoside linkages can also be used in the present invention. Such modified backbones include ones that have a phosphorus atom in the backbone and others that do not have a phosphorus atom in the backbone. Examples of modified linkages include, but are not limited to, phosphorothioate and 5′-N-phosphoramidite linkages. See Kornberg and Baker, DNA Replication, 2nd ed., Freeman, San Francisco, 1992; Scheit, Nucleotide Analogs, John Wiley, New York, 1980; U.S. Patent Pub. No. 20040092470 and references therein for further discussion of various nucleotides, nucleosides, and backbone structures that can be used in the polynucleotides or oligonucleotides described herein, and methods for producing them.

Polynucleotides and oligonucleotides need not be uniformly modified along the entire length of the molecule. For example, different nucleotide modifications, different backbone structures, etc., may exist at various positions in the polynucleotide or oligonucleotide. Any of the polynucleotides described herein may utilize these modifications.

The polynucleotide may be provided by any means known in the art. In certain embodiments, the polynucleotide has been engineered using recombinant techniques. See Ausubel, et al., Current Protocols in Molecular Biology, Wiley, 1999; Molecular Cloning: A Laboratory Manual, 2nd ed., ed. by Sambrook, Fritsch, and Maniatis, Cold Spring Harbor Laboratory Press, 1989. The polynucleotide may also be obtained from natural sources and purified from contaminating components found normally in nature. The polynucleotide may be synthesized using enzymatic techniques, either within cells or in vitro. The polynucleotide may also be chemically synthesized in a laboratory, e.g., using standard solid phase chemistry. The polynucleotide may be modified by chemical and/or biological means. In certain preferred embodiments, these modifications lead to increased stability of the polynucleotide. Modifications include methylation, phosphorylation, end-capping, etc.

A nucleic acid sequence is presented in the 5′ to 3′ direction unless otherwise indicated.

The term “pore forming agent” refers to a broad class of volatile materials that are used during the process to create porosity in the resultant matrix. The pore forming agent can be a volatilizable solid or liquid such as ammonium acetate, ammonium chloride, methylene chloride, pentane, and toluene.

The phrase “sense region” refers to a sequence of nucleotides in a polynucleotide that is complementary to an antisense region in the same polynucleotide (if the polynucleotide is a unimolecular polynucleotide having both a sense and antisense sequence, wherein the sense and antisense sequences are capable of annealing by reason of the polynucleotide forming intramolecular interactions such as, for example, a hairpin structure), or in a different polynucleotide (in the case of a double stranded polynucleotide that comprises two separate strands, one bearing a sense sequence and one bearing an antisense sequence, wherein the sense and antisense sequences are capable of annealing by reason of the two strands undergoing an intermolecular interaction to form, for example, a duplex). Typically, an mRNA sequence corresponds to the sense sequence, as it is the sequence that is translated into protein by the ribosome.

The term “siRNA” refers to small inhibitory RNA duplexes that induce the RNA interference (RNAi) pathway. These molecules can vary in length (generally between 18-30 base pair) and contain varying degrees of complementarity to their target mRNA in the antisense strand. Some, but not all siRNA have unpaired, overhanging bases on the 5′ or 3′ end of the sense strand and/or the antisense strand. An siRNA molecule can be bimolecular, such as separate sense and antisense strands annealed through non-covalent interaction, or can be unimolecular, as when sense and antisense strands are regions of a hairpin structure that comprises a loop structure and, optionally, a stem region and/or terminal structure.

“Target mRNA” refers to a messenger RNA to which a given siRNA can be directed against. “Target sequence” and “target site” refer to a sequence within the mRNA to which the sense strand of an siRNA shows varying degrees of homology and the antisense strand exhibits varying degrees of complementarity. The term “siRNA target” can refer to the gene, mRNA, or protein against which an siRNA is directed. Similarly “target silencing” can refer to the state of a gene, or the corresponding mRNA or protein.

“Tripeptide,” also referred to herein as a trimer, refers to a peptide composed of three amino acids.

“Volume median diameter” or “VMD” is a measure of mean particle size, defined by a volume distribution of particle sizes. The VMD is calculated by multiplying each particle diameter by the volume of all particles of that size and summing. This is then divided by the total volume of all particles.

Active RNAi Agents

Active agents for incorporation in the compositions of this invention are oligonucleotide(s) including siRNAs, shRNAs, and precursors thereof, which are collectively described herein as “siNAs.”

The length of the duplex region of an siRNA can range from 18 to 31 base pairs (bp), or from 18 to 26 bp, or from 19 to 23 bp.

An siRNA can have an overhang on either end of the duplex region. The overhang can be on the 5′ or 3′ end of the sense and/or antisense strand. An overhang may be from 1 to 5 nucleotides (nt) in length, or longer. Often, an overhang is on the 3′ end of the sense and/or antisense strand.

In addition, the antisense strand or the strand designed to bind/anneal to the target (i.e., the guide strand) has substantial complementarity to the target. The guide strand may have greater than 79% complementarity with the target, or greater than 84% complementarity with the target, or greater than 89% complementarity with the target. The guide strand may have greater than 95% complementarity with the target.

The oligonucleotide(s) may be synthetic in nature and as such can be generated by a range of chemistries (e.g., ACE chemistry) recognized in the art of nucleic acid synthesis. Alternatively, the siRNA may be generated by enzymatic means (e.g., nuclease cleavage, in vitro or in vivo transcription, PCR, etc.).

In addition, the oligonucleotide(s) can contain chemical modifications and/or conjugates. Such modifications and/or conjugates can be associated with the base, the sugar, or the internucleotide region, and can be added to enhance siRNA stability, specificity, and/or deliverability to the cell type(s) of interest. Modifications and/or conjugates can include small molecules, peptides, polypeptides, proteins, simple sugars, di- or tri-saccharides, polysaccharides, various polymers, steroids, nucleotides, oligonucleotides, polynucleotides, fats, and the like.

The active RNA agent can be a pooling of siNAs. The active RNA agent may be a homogeneous or heterogeneous population of siNAs. In cases where the pooled siNAs are heterogeneous, the pool can target multiple sites of a single gene transcript, or target two or more genes.

The active RNA agent, when administered by inhalation, intranasal, or pulmonary delivery may act locally or systemically, so that the amount of active agent in the formulation is an amount necessary to deliver a therapeutically effective amount of the active agent to achieve the desired result. In practice, the therapeutically effective amount may vary, depending upon the agent, its activity, the severity of the condition to be treated, the patient population, dosing requirements, and the desired therapeutic effect.

The compositions and formulations of the RNAi agent will generally contain from about 0.1% by weight to about 99% by weight active agent, or from about 2% to about 95% by weight active agent, or from about 5% to 85% by weight active agent, or from about 10% to 30% by weight active agent, and will also depend upon the relative amounts of additives, carriers, and/or excipients contained in the composition.

The compositions of the invention are particularly useful for active agents that are delivered in doses of from 0.001 mg/kg/day to 100 mg/kg/day, or in doses from 0.01 mg/kg/day to 75 mg/kg/day, or in doses from 0.10 mg/kg/day to 50 mg/kg/day, or in doses of from 5 mg/kg/day to 20 mg/kg/day.

Nucleic acid agents useful for this invention may be single-stranded nucleic acids, double-stranded nucleic acids, or modified or degradation-resistant nucleic acids.

In this context, this invention provides compositions, formulations and methods for modulating gene expression by RNA Interference. A composition or formulation of this invention may release a ribonucleic acid agent to a cell which can produce the response of RNAi. Compositions or formulations of this invention may release ribonucleic acid agents to a cell upon contact with an intracellular endosome. The release of a ribonucleic acid agent intracellularly may provide inhibition of gene expression in the cell.

A siRNA of this invention may have a sequence that is complementary to a region of a viral gene. For example, some compositions and methods of this invention are useful to regulate expression of the viral genome of an influenza.

In this context, this invention provides compositions and methods for modulating expression and infectious activity of an influenza virus by RNA Interference. Expression and/or activity of an influenza can be modulated by delivering to a cell, for example, a short interfering RNA molecule having a sequence that is complementary to a region of a RNA polymerase subunit of an influenza. For example, in Table 1 are shown double-stranded siRNA molecules with sequence homology to an RNA polymerase subunit of an influenza.

TABLE 1

Double-Stranded siRNA Molecules
Targeted to Influenza

| | | |
|---|---|---|
| | Sub- | |
| siRNA | unit | SEQUENCE |

| | | | |
|---|---|---|---|
| G3789 | PB2 | (SEQ ID NO 1) | CGGGACUCUAGCAUACUUAdTdT |
| | | (SEQ ID NO 2) | UAAGUAUGCUAGAGUCCCGdTdT |
| G3807 | PB2 | (SEQ ID NO 3) | ACUGACAGCCAGACAGCGAdTdT |
| | | (SEQ ID NO 4) | UCGCUGUCUGGCUGUCAGUdTdT |
| G3817 | PB2 | (SEQ ID NO 5) | AGACAGCGACCAAAAGAAUdTdT |
| | | (SEQ ID NO 6) | AUUCUUUUGGUCGCUGUCUdTdT |
| G6124 | PB1 | (SEQ ID NO 7) | AUGAAGAUCUGUUCCACCAdTdT |
| | | (SEQ ID NO 8) | UGGUGGAACAGAUCUUCAUdTdT |
| G6129 | PB1 | (SEQ ID NO 9) | GAUCUGUUCCACCAUUGAAdTdT |
| | | (SEQ ID NO 10) | UUCAAUGGUGGAACAGAUCdTdT |
| G8282 | PA | (SEQ ID NO 11) | GCAAUUGAGGAGUGCCUGAdTdT |
| | | (SEQ ID NO 12) | UCAGGCACUCCUCAAUUGCdTdT |
| G8286 | PA | (SEQ ID NO 13) | UUGAGGAGUGCCUGAUUAAdTdT |
| | | (SEQ ID NO 14) | UUAAUCAGGCACUCCUCAAdTdT |
| G1498 | NP | (SEQ ID NO 15) | GGAUCUUAUUUCUUCGGAGdTdT |
| | | (SEQ ID NO 16) | CUCCGAAGAAAUAAGAUCCdTdT |

A siRNA of this invention may have a sequence that is complementary to a region of a RNA polymerase subunit of an influenza.

This invention provides compositions and methods to administer siNAs directed against a mRNA of an influenza, which effectively down-regulates an influenza RNA and thereby reduces, prevents, or ameliorates an influenza infection.

Pharmaceutical Compositions and Formulations

The compositions and formulations of this invention may include one or more pharmaceutical excipients which are suitable for pulmonary administration. These excipients, if present, are generally present in the composition in amounts ranging from about 0.01% to about 95% percent by weight, and more preferably from about 0.5 to about 80%. Preferably, such excipients serve to improve the features of the active agent composition, e.g., by providing more efficient and reproducible delivery of the active agent, improving the handling characteristics of powders (e.g., flowability and consistency), the stability of the agent, and/or facilitating manufacturing and filling of unit dosage forms. In particular, excipient materials function to further improve the physical and chemical stability of the active agent, aid in integration of the particle into the pulmonary mucosal layer, and enhance transfection of the active agent into the cell, thus increasing efficacy of the active agent, minimize the residual moisture content and hinder moisture uptake, and to enhance particle size, degree of aggregation, particle surface properties (i.e., rugosity), ease of inhalation, and the targeting of particles to the lung. The excipient(s) may also serve simply as bulking agents when it is desired to reduce the concentration of active agent in the formulation.

Within the compositions, formulations and methods of this invention, the active agent may be combined or coordinately administered with a suitable carrier or vehicle. As used herein, the term “carrier” means a pharmaceutically acceptable solid or liquid filler, diluent or encapsulating or carrying material.

A carrier can contain pharmaceutically acceptable additives such as acidifying agents, alkalizing agents, antimicrobial preservatives, antioxidants, buffering agents, chelating agents, complexing agents, solubilizing agents, humectants, solvents, suspending and/or viscosity-increasing agents, tonicity agents, wetting agents or other biocompatible materials. Examples of ingredients, pharmaceutical excipients and/or additives of the above categories suitable for use in the compositions and formulations of this invention can be found in the U.S. Pharmacopeia National Formulary, 1990, pp. 1857-1859, as well as in Raymond C. Rowe, et al., Handbook of Pharmaceutical Excipients , 5th ed., 2006, and “Remington: The Science and Practice of Pharmacy,” 21st ed., 2006, editor David B. Troy, and in the Physician's Desk Reference, 52nd ed., Medical Economics, Montvale, N. J., 1998.

Some examples of the materials which can serve as pharmaceutically acceptable carriers are sugars, such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols, such as propylene glycol; polyols such as glycerin, sorbitol, mannitol and polyethylene glycol; esters such as ethyl oleate and ethyl laurate; agar; buffering agents such as magnesium hydroxide and aluminum hydroxide; alginic acid; pyrogen free water; isotonic saline; Ringer's solution, ethyl alcohol and phosphate buffer solutions, as well as other non toxic compatible substances used in pharmaceutical formulations. Wetting agents, emulsifiers and lubricants such as sodium lauryl sulfate and magnesium stearate, as well as coloring agents, release agents, coating agents, sweetening, flavoring and perfuming agents, preservatives and antioxidants can also be present in the compositions, according to the desires of the formulator. Examples of pharmaceutically acceptable antioxidants include water soluble antioxidants such as ascorbic acid, cysteine hydrochloride, sodium bisulfite, sodium metabisulfite, sodium sulfite and the like; oil-soluble antioxidants such as ascorbyl palmitate, butylated hydroxyanisole (BHA), butylated hydroxytoluene (BHT), lecithin, propyl gallate, alpha-tocopherol and the like; and metal-chelating agents such as citric acid, ethylenediamine tetraacetic acid (EDTA), sorbitol, tartaric acid, phosphoric acid and the like.

Pharmaceutical excipients useful in the present composition include but are not limited to amino acids, peptides, proteins, non-biological polymers, biological polymers, simple sugars, carbohydrates, and salts which may be present singly or in combination. Also preferred excipients have glass transition temperatures (Tg) above about 35° C., or above about 40° C., or above 45° C., or above about 55° C. This temperature is important in creating a stable product as well as having desirable aerosol properties of the dry powder.

Proteins and peptides may be desirable components of the formulation because they promote cell fusion, dispersion, and uptake of the active agent. Exemplary protein excipients include albumins such as human serum albumin (HSA), recombinant human albumin (rHA), gelatin, casein, hemoglobin, hemagglutinin, and other fusion proteins (such as those encoded by viruses (e.g., HIV) and the like).

Further, exemplary peptides include sequences that are derived from proteins that participate in fusion (e.g., hemagglutinin fusion peptide, Lague, P., et al., J. Mol. Biol. 354(5):1129-41, Dec. 16, 2005, or can comprise poly amino acids such as poly leucine. Dispersibility-enhancing peptide excipients include dimers, trimers, tetramers, and pentamers comprising one or more hydrophobic amino acid components. Amino acids that fall into this category include hydrophobic amino acids such as leucine, valine, isoleucine, tryptophan, alanine, methionine, phenylalanine, tyrosine, histidine, and proline.

The formulation may also comprises amino acids because they promote cell fusion, can act as a bulking agent, enhance dispersability, and can negate the negative charge associated with the siRNA. Suitable amino acids which may function in a buffering capacity, dispersing agents, transfection agent, bulking agent, negate siRNA charge in dry powder, and promote cell fusion include alanine, glycine, arginine, betaine, histidine, glutamic acid, aspartic acid, cysteine, lysine, leucine, isoleucine, valine, methionine, phenylalanine, aspartame, tyrosine, tryptophan, and the like. Amino acids that enhance dispersion include hydrophobic amino acids such as leucine, valine, isoleucine, tryptophan, alanine, methionine, phenylalanine, tyrosine, and proline. In some embodiments, peptides used in the formulation are arginine and leucine.

The formulation optionally comprises sugars that can act as bulking agents, enhance cell targeting (e.g., galactose and lactose), open cellular junctions (e.g., mannitol), and improve particle flight properties by altering particle density. Carbohydrate excipients suitable for use in the invention include, for example, monosaccharides such as fructose, maltose, galactose, glucose, D-mannose, sorbose, and mixtures thereof, disaccharides, such as lactose, sucrose, trehalose, cellobiose, and mixtures thereof, polysaccharides, such as raffinose, melezitose, maltodextrins, dextrans, starches, and mixtures thereof; and alditols, such as mannitol, xylitol, maltitol, lactitol, xylitol sorbitol (glucitol), pyranosyl sorbitol, myoinositol and mixtures thereof. Sugars that can be used in a formulation of this invention include lactose and sucrose.

The formulation may include lipids that can serve a number of roles including acting as transfection or complexation agents, and incorporate into the mucusilliary layer. In addition, lipids can act as the shell of the active agent particle and play a role in determining particle size. Lipid excipients suitable for use in the invention include, for example, cationic lipids such as dipalmitoylethylphosphocholine (DpePC), Dioleoyl phosphatidylethanolamine (DOPE), 3β-[N-(N′,N′-Dimethylaminoethane)-carbamoyl]Cholesterol (DC cholesterol), and mixtures thereof, anionic lipids such as 1,2-Dioleoyl-sn-Glycero-3-[Phospho-L-Serine] (Sodium Salt)(DOPS),1,2-Dioleoyl-sn-Glycero-3-Phosphate (Monosodium Salt)(DOPA), and mixtures thereof, non-ionic lipids such as 1,2-Dipalmitoyl-sn-Glycero-3-Phosphocholine (DPPC), distearoylphosphatidylcholine (DSPC), diarachidoylphosphatidylcholin (DAPC), dipalmitoyl phosphatidylethanolamine (DPPE) and mixtures thereof; and fatty acids, such as Oleic acid, myristoleic, aracadonic acid and mixtures thereof. A lipid used in the formulations of the invention may be DPPC.

The compositions may also include a buffer or a pH adjusting agent, typically a salt prepared from an organic or inorganic acid or base. Salts that can be used in the invention can complex with the active agent to form precipitates, can increase yields of the process, aid in transfection of the active agent into the cell, and alter the overall density of the powder. Representative buffers include acid salts of citric acid, ascorbic acid, gluconic acid, carbonic acid, tartaric acid, succinic acid, acetic acid, or phthalic acid, Tris, tromethamine hydrochloride, or phosphate buffers. The buffer adjusting agent may be calcium chloride, sodium citrate, protamine sulfate, sodium chloride, calcium phosphate, or mixtures thereof. Such salts can be employed to adjust the pH or osmolarity of the formulation.

The compositions of this invention may also include polymeric excipients/additives. Polymers can complex with the active agent and enhance transfection into the cell. In addition, polymers can modulate the release of the active agent, and mask particles, thus enhancing the bioavailability and/or half life of the active agent. Polymers can also enhance binding of particles to targeting moieties and promote cell fusion. Exemplary polymers include polyvinylpyrrolidones, derivatized celluloses such as hydroxymethylcellulose, hydroxyethylcellulose, and hydroxypropylmethylcellulose, Ficolls (a class of polymeric sugars), hydroxyethylstarch, dextrates (e.g., cyclodextrins, such as 2-hydroxypropyl-β-cyclodextrin and sulfobutylether-β-cyclodextrin), polyethylene glycols, and pectin. Additional polymers include poly(lactide-co-glycolide) (PLGA), polylactide (PLA), polyethylene imine (PEI), poly-L-lysine (PLL) and other cationic polymers.

The compositions may optionally comprise flavoring agents, taste-masking agents, inorganic salts (e.g., sodium chloride), antimicrobial agents (e.g., benzalkonium chloride), sweeteners, antioxidants, antistatic agents, surfactants (e.g., polysorbates such as “TWEEN 20” and “TWEEN 80”), sorbitan esters, lipids (e.g., phospholipids such as lecithin and other phosphatidylcholines, phosphatidylethanolamines), fatty acids and fatty esters, steroids (e.g., cholesterol), and chelating agents (e.g., EDTA, zinc and other such suitable cations).

Preparing Dry Powders

Dry powder formulations may be prepared by spray drying. Spray drying of the formulations can be carried out, for example, as described generally in the Spray Drying Handbook, 5th ed., K. Masters, John Wiley & Sons, Inc., NY, N.Y., 1991, and in Platz, R., et al., International Patent Publication No. WO 97/41833, 1997.

The pre-spray dried solutions will generally contain solids dissolved at a concentration from 0.01% (weight/volume) to about 20% (weight/volume), or from 0.1% to 3% (weight/volume). All of the reagents used in this process must be of sufficient quality to avoid degradation of the active agent under ambient conditions.

In one instance, active agents can be sprayed dried from an aqueous solution. Utilizing this non-limiting approach, the active agent is first dissolved in water, optionally containing a physiologically acceptable buffers, proteins, peptides, amino acids, carbohydrates, simple sugars, and/or water soluble polymers of the invention. The pH range of active agent-containing solutions is generally from about 2 to about 9, or from 6 to about 8.

More preferably, formulations comprised of water soluble excipients (e.g., sugars, salts, amino acids, water soluble polymers, water soluble proteins, water soluble emulsifiers and/or surfactants, ammonium bicarbonate and/or other pore forming agents, peptides), water soluble active agents (e.g., siRNA), and non-water soluble excipients (e.g., neutral lipids, cationic lipids, anionic lipids, non-water soluble polymers and non-soluble emulsifiers) are first weighed out in separate containers. Contaminant free water or water containing a suitable salt or buffer is then added to the siRNA and water-soluble excipient containers, and organic solvents (e.g., ethanol, methanol, isopropanol, acetone, methylene chloride, toluene, hexane, ethylacetate, and others) are added to the non-water soluble excipients. The appropriate amount of each active agent (in water) is then added to the aqueous phase containing water soluble excipients. The resulting aqueous solution containing the active agent and water soluble excipients is then combined with the organic phase containing non-water soluble excipients. Depending upon the organic solvent being used, this resulting formulation may or may not need to be continually stirred and/or homogenized, depending upon whether the aqueous and organic solutions are miscible. Preferred solvents include acetone, alcohols and the like. Representative alcohols are lower alcohols such as methanol, ethanol, propanol, isopropanol, and mixtures thereof. When formulations demand mixing of aqueous and organic phases, mixing typically occurs under conditions where the temperature is maintained at approximately 25° C. and mixing occurs by stirplate.

In some embodiments, the dry powder formulation comprises siRNA, DPPC, sucrose, and albumin (20:40:20:20 by weight). To prepare these lots, an aqueous solution containing siRNA, albumin, and sucrose can be mixed with ethanol containing DPPC and then spray dried under conditions where Tᵢₙₗₑₜ=95° C., Tₒᵤₜₗₑₜ=˜55° C., with an atomization/drying gas flow rate of about 600 L/hr.

In some embodiments, the dry powder formulation comprises siRNA, DPPC, lactose, and protamine (20:45:30:5 by weight). To prepare these lots, an aqueous solution containing siRNA, protamine sulfate, and lactose can be mixed with ethanol containing DPPC. The mixture can be spray dried under conditions where Tᵢₙₗₑₜ=95° C., Tₒᵤₜₗₑₜ=˜55° C., with an atomization/drying gas flow rate of about 600 L/hr.

In some embodiments, the dry powder formulation can comprise siRNA, DPPC, lactose, and arginine (20:45:30:5 by weight). To prepare these lots, an aqueous solution containing siRNA, arginine, and lactose can be mixed with ethanol containing DPPC. After the aqueous solution is added to the organic solution the mixture can be spray dried under conditions where Tᵢₙₗₑₜ=95° C., Tₒᵤₜₗₑₜ=˜55° C., with an atomization/drying gas flow rate of about 600 L/hr.

In some embodiments, the dry powder formulation may comprise siRNA, DPPC, lactose, and calcium chloride (20:47:30:3 by weight). To prepare these lots, an aqueous solution containing siRNA, calcium chloride, and lactose may be mixed with ethanol containing DPPC. The mixture may then be spray dried under conditions where Tᵢₙₗₑₜ=95° C., Tₒᵤₜₗₑₜ=˜55° C., with an atomization/drying gas flow rate of about 600 L/hr.

In some embodiments, the dry powder formulation may comprise siRNA, DPPC, leucine, and calcium chloride (20:47:30:3 by weight). To prepare these lots, an aqueous solution containing siRNA, calcium chloride, and lactose may be mixed with ethanol containing DPPC. The mixture may then be spray dried under conditions where Tᵢₙₗₑₜ=95° C., Tₒᵤₜₗₑₜ=˜50° C., with an atomization/drying gas flow rate of about 600 L/hr.

In some embodiments, the siRNA can be prepared as a particulate prior to preparation of the dry powder. Preparing the active agent in this way ensures that submicron-size particles containing the siRNA are first formed. The active agent can be induced to form a particulate by a variety of methods known to those skilled in the art. In some embodiments, an aqueous solution of siRNA is mixed with a salt (e.g., sodium chloride, calcium chloride, calcium phosphate) and added to an organic solvent (e.g., ethanol) such that the siRNA precipitates as fine particles. If ethanol is used, the final ethanol concentration may be 60-80%. The conditions for precipitation will influence the size of the particles, and manipulation of conditions (for example, but not limited to, time, temperature, stirring rate, and presence and concentrations of surfactants, lipids, polycations, and other excipients) will produce particles of various sizes. In some embodiments, the particles are less than 300 nm in diameter in their longest dimension. Upon spray drying, these particles will be incorporated into larger dry powder particles with the aerodynamic properties suitable for pulmonary delivery described herein.

In some embodiments, a dry powder formulation may comprise siRNA, DPPC, lactose, and calcium chloride (20:47:30:3 by weight). An aqueous solution containing siRNA and calcium chloride may be mixed with ethanol and incubated overnight at −20° C. The next day a defined amount of lactose may be dissolved in nuclease free water and DPPC dissolved in ethanol. The aqueous phase may then be added to the organic phase and the precipitated siRNA solution added to this mixture. Afterward, the solutions may be spray dried under conditions where Tᵢₙₗₑₜ=95° C., Tₒᵤₜₗₑₜ=˜50° C., with an atomization/drying gas flow rate of about 600 L/hr.

In some embodiments, the siRNA and other water soluble excipients can be encapsulated within a non water soluble shell. The aqueous phase containing the water soluble excipients can be emulsified with a non-water miscible organic solvent. The resulting water in oil emulsion may then be added to a second aqueous phase that may or may not contain additional excipients. The emulsion and aqueous phase can then be emulsified creating a water in oil in water emulsion. The resulting emulsion is then spray dried into particles suitable for pulmonary delivery as described herein.

In some embodiments, a dry powder formulation may comprise siRNA, DPPC, lactose, and calcium chloride (20:47:30:3 by weight). An aqueous solution containing siRNA, lactose, and calcium chloride may be mixed with a solution of methylene chloride and DPPC. The mixture may then be emulsified creating a water in oil emulsion. The water in oil emulsion may then be added to a second aqueous solution containing no excipients. The secondary mixture may then be emulsified and spray dried under conditions where Tᵢₙₗₑₜ95° C., Tₒᵤₜₗₑₜ=˜50° C., with an atomization/drying gas flow rate of about 600 L/hr.

The formulations can be spray dried in a conventional spray drier, such as those available from commercial suppliers (for example Niro A/S, Denmark, Buchi, Switzerland) resulting in a dispersible, dry powder.

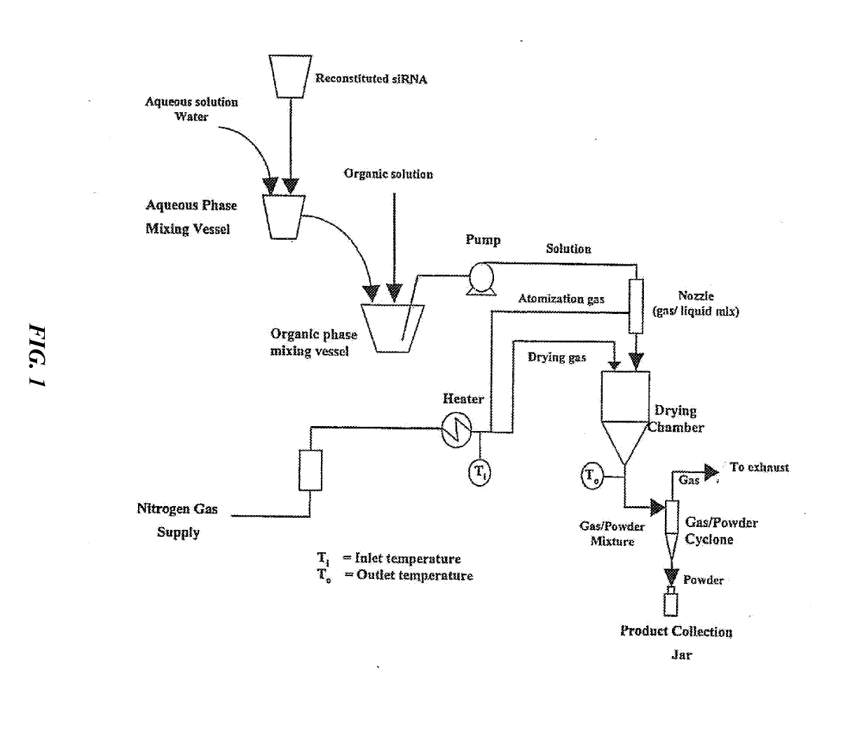

FIG. 1 shows an example of a dry powder manufacturing process.

The gas used to spray dry the material is typically dry nitrogen, although inert gases such as argon are also suitable. Moreover, the temperature of both the inlet and outlet of the gas used to dry the sprayed material is such that it does not cause decomposition of the active agent in the sprayed material. Such temperatures are typically determined experimentally, although generally, the inlet temperature will range from about 65° C. to about 125° C. while the outlet temperature will range from about 30° C. to about 70° C. Once again, all of the materials used in this process must be of sufficient quality to avoid degradation of the active agent.

In some embodiments, the dry powder can be prepared by combining an aqueous solution containing a predetermined amount of active agent with desired excipients, with a predetermined volume of organic solution containing the desired excipients. Subsequently, the formulation can be spray dried under conditions where Tᵢₙₗₑₜ=95° C., Tₒᵤₜₗₑₜ=˜55° C., with an atomization/drying gas flow rate of about 600 L/hr.

Alternatively, powders may be prepared by lyophilization, vacuum drying, spray freeze drying, super critical fluid processing, air drying, or other forms of evaporative drying. In some instances, it may be desirable to provide the dry powder formulation in a form that possesses improved handling/processing characteristics, e.g., reduced static, better flowability, low caking, and the like, by preparing compositions composed of fine particle aggregates, that is, aggregates or agglomerates of the above-described dry powder particles, where the aggregates are readily broken back down to the fine powder components for pulmonary delivery, as described, for example, U.S. Pat. No. 5,654,007.

In another approach, dry powders may be prepared by agglomerating the powder components, sieving the materials to obtain agglomerates, spheronizing to provide a more spherical agglomerate, and sizing to obtain a uniformly-sized product, as described, for example, in PCT International Publication No. WO 95/09616.

Dry powders may also be prepared by blending, grinding, sieving or jet milling formulation components in dry powder form.

Once formed, the dry powder compositions may be maintained under dry (i.e., relatively low humidity) conditions during manufacture, processing, and storage. The dry powder compositions and formulations of this invention can be stored under conditions whereby the temperature is from 2 to 8 degrees Celsius and the relative humidity is less than 30%.

Dry Powder Formulations

Powders of this invention may have (i) consistently high dispersivities, which are maintained, even upon storage, (ii) small aerodynamic particles sizes (MMADs), and/or (iii) improved fine particle dose values, i.e., powders having a high percentage of particles sized less than 5.6 microns MMAD.

Dry powders of this invention may be composed of aerosolizable particles that effectively penetrate into the lungs. The particles of this invention may have a mass median diameter (MMD) of less than about 18 μm, or less than about 15 μm, or less than about 13 μm, or less than about 10 μm, or in the range of 0.7 μm to 10 μm in diameter. Powders can be composed of particles having an MMD of about 1.5 to 5.5 μm.

The powders of this invention may have an aerosol particle size distribution less than about 8 μm mass median aerodynamic diameter (MMAD), or less than 6 μm. The mass median aerodynamic diameters of the powders may range from about 1-6 μm.

Particle size measurements can be made with a Rodos/Helos particle size laser diffraction analyzer. One to five milligrams of the dry powder is placed into the inlet on the Helos dry particle size hopper. The particle sizer disperses the dry powder, and a particle size is measured. The experiment is repeated 3 times and an average particle size is taken. The dispersion forces on the dry powder disperser are more efficient than the dispersion forces observed during in-vivo dosing of the mice using an insufflator (Penn Century, Philadelphia, Pa.).

The mass median diameters (MMD) of the powders can be calculated using a Rodos/Helos particle size laser diffraction analyzer and the density of the particle.

The powders of this invention may further be characterized by their densities. A powder may possess a bulk density from about 0.04 to about 2 g/cubic centimeter.

The powders will generally have a moisture content below about 10% by weight, or below about 5% by weight, or below about 3% by weight.

The compositions of this invention may have dispersibility, as indicated by the delivery efficiency value. The mean delivery efficiency (DE) of dry powders may be greater than 30%, or greater than 40%, or greater than 50%, or greater than 60%.

An additional measure for characterizing the overall aerosol performance of a dry powder is the fine particle fraction (FPF), which describes the percentage of powder having an aerodynamic diameter less than 5.6 microns. The powders of this invention may have FPF values ranging from about 20 to about 70%.

The compositions can formulations of this invention can have good stability, with respect to both chemical stability and physical stability, i.e., aerosol performance, over time. With respect to chemical stability, the active agent contained in the formulation may degrade by no more than about 10% over a time course of 18 months.

With respect to aerosol performance, compositions and formulations of this invention may exhibit a drop in emitted dose of no more than about 20%, or no more than about 10%, or no more than about 5%, when stored under ambient conditions for a period of three months.

The improvement in aerosol properties can result in several related advantages, such as: (i) reducing costly drug loses to the inhalation device, since more powder is aerosolized and is therefore available for inhalation by a subject; (ii) reducing the amount of dry powder required per unit dose, due to the high efficiency of aerosolization of powder, and/or (iii) reducing the number of inhalations per day by increasing the amount of aerosolized drug reaching the lungs of a subject (as compared to treatments with the active agent alone).

In cases where the target of the active RNAi agent is an infectious agent such as a virus, an additional measure for judging the overall performance of a dry powder involves measuring the effect of agent delivery in the formulation on viral titer. To accomplish this, test animals (e.g., mice) may be exposed to the formulation containing the agent, preceded by, or followed by exposure to the virus. After a sufficient period, the animal can be sacrificed and the pulmonary tissues removed. The tissues can then be homogenized, and the resultant viral titer measured using art-proven techniques (e.g., TCID₅₀ assay).

Where the target of the active agent is an endogenous, host-encoded gene, an additional measure for characterizing the overall performance of a dry powder involves measuring the effect of agent delivery on gene knockdown. To accomplish this, test animals (e.g., mice) can exposed to a formulation containing the agent(s). After a sufficient period, the animal can be sacrificed and the pulmonary tissues removed. The tissues can then be homogenized, RNA extracted, and the resultant expression of the transcript of interest determined using various techniques (e.g., RT-PCR, Branched-DNA assays).

Administration

The compositions and formulations of this invention may be delivered using any suitable dry powder inhaler (DPI), i.e., an inhaler device that utilizes the patient's inhaled breath as a vehicle to transport the dry powder to the lungs.

When administered using a device of this type, the powder may be contained in a receptacle having a puncturable lid or other access surface, or a blister package or cartridge, where the receptacle may contain a single dosage unit or multiple dosage units. Methods for filling large numbers of cavities (i.e., unit dose packages) with metered doses of dry powder medicament are described, for example, in WO 97/41031.

Also suitable for delivering the powders described herein are dry powder inhalers of the type described, for example, in U.S. Pat. No. 3,906,950 and in U.S. Pat. No. 4,013,075, wherein a premeasured dose of dry powder for delivery to a subject is contained within a hard gelatin capsule.

Other dry powder dispersion devices for administering dry powders to the pulmonary tissues include those described, for example, in Newell, R. E., et al., European Patent No. EP 129985, 1988; in Hodson, P. D., et al., European Patent No. EP 472598, 1996; in Cocozza, S., et al., European Patent No. EP 467172, 1994, and in Lloyd, L. J., et al., U.S. Pat. No. 5,522,385, 1996.

Also suitable for delivering the dry powders of this invention are inhalation devices such as the Astra-Draco “TURBUHALER.” This type of device is described in detail in Virtanen, R., U.S. Pat. No. 4,668,281, 1987; in Wetterlin, K., et al., U.S. Pat. No. 4,667,668, 1987; and in Wetterlin, K., et al., U.S. Pat. No. 4,805,811, 1989.

Other suitable devices include dry powder inhalers such as the Rotahaler.RTM. (Glaxo), Discus.RTM. (Glaxo), Spiros.RTM. inhaler (Dura Pharmaceuticals), and the Spinhaler.RTM. (Fisons).

Also suitable are devices which employ the use of a piston to provide air for either entraining powdered medicament, lifting medicament from a carrier screen by passing air through the screen, or mixing air with powder medicament in a mixing chamber with subsequent introduction of the powder to the patient through the mouthpiece of the device, such as described in U.S. Pat. No. 5,388,572.

Dry powders may also be delivered using a pressurized, metered dose inhaler (MDI), e.g., the Ventolin.RTM metered dose inhaler, containing a solution or suspension of drug in a pharmaceutically inert liquid propellant, e.g., a chlorofluorocarbon or fluorocarbon, as described in U.S. Pat. No. 5,320,094, and in U.S. Pat. No. 5,672,581.

Alternatively, powders may be dissolved or suspended in a solvent, e.g., water, ethanol, or saline, and administered by nebulization. Nebulizers for delivering an aerosolized solution include the AERx™ (Aradigm), the Ultravent.RTM. (Mallinkrodt), and the Acorn II.RTM. (Marquest Medical Products).

Prior to use, dry powders can be stored under ambient conditions, and may be stored at temperatures at or below about 25° C., and relative humidities (RH) ranging from about 15 to 80%, or less than about 40%, using a dessicating agent in the secondary packaging of the dosage form.

All publications, books, references, patents, patent publications and patent applications cited herein are each hereby specifically incorporated by reference in their entirety.

While this invention has been described in relation to certain embodiments, and many details have been set forth for purposes of illustration, it will be apparent to those skilled in the art that this invention includes additional embodiments, and that some of the details described herein may be varied considerably without departing from this invention. This invention includes such additional embodiments, modifications and equivalents. In particular, this invention includes any combination of the features, terms, or elements of the various illustrative components and examples.

The use herein of the terms “a,” “an,” “the,” and similar terms in describing the invention, and in the claims, are to be construed to include both the singular and the plural. The terms “comprising,” “having,” “including,” and “containing” are to be construed as open-ended terms which mean, for example, “including, but not limited to.” Recitation of a range of values herein refers individually to each separate value falling within the range as if it were individually recited herein, whether or not some of the values within the range are expressly recited. Specific values employed herein will be understood as exemplary and not to limit the scope of the invention.

Definitions of technical terms provided herein should be construed to include, without recitation, those meanings associated with these terms known to those skilled in the art, and are not intended to limit the scope of the invention.

The examples given herein, and the exemplary language used herein are solely for the purpose of illustration, and are not intended to limit the scope of the invention.

When a list of examples is given, such as a list of compounds or molecules suitable for this invention, it will be apparent to those skilled in the art that mixtures of the listed compounds or molecules are also suitable.

### EXAMPLES

### Example 1

### Materials

Ethanol, Denatured, anhydrous (VWR International, West Chester, Pa.).
Sodium citrate (USP, Sigma Aldrich Inc., St. Louis, Mo.).
Calcium chloride dihydrate (Certified ACS, Fisher Scientific Company L.L.C., Fair Lawn, N.J.).
Albumin from bovine serum, minimum 98% (Sigma Aldrich Inc., St. Louis, Mo.).
1,2-Dipalmitoyl-sn-Glycero-3-Phosphocholine (DPPC)(Genzyme Corporation, Cambridge, Mass.).
D-(+)-lactose, monohydrate (ACS Reagent, JT Baker, Phillipsburg, N.J.).
L-Arginine (≧99.5% (NT), Fluka AG, Switzerland).
L-Leucine (≧99.5% (NT), Fluka AG, Switzerland).
Sucrose (Analytical Reagent, Mallinckrodt Baker, Paris KY.).
Protamine sulfate from salmon (Grade X, Sigma Aldrich Inc., St. Louis, Mo.).
Influenza: Strain PR8.

### Example 2

### Method for Determining Viral Titer by Hemagglutination Assay

Viral titering was used to determine the effectiveness of various formulations of the invention for siRNA delivery. Specifically, for prophylacetic use, siRNA targeting the influenza virus nucleoprotein mRNA were formulated into dry powder formulations and administered (10 mg/kg siRNA) to Balb/c mice intranasally or intratracheally. Animals were anesthetized with a mixture of ketamine and xylazine. Four hours later, mice were inoculated (intranasally) with 30 PR8 viral influenza particles to initiate infection. Mice were sacrificed at 48 h following infection, and lungs were harvested. Lungs were homogenized, and the homogenate was frozen and thawed twice to release virus.

The siRNA was G1498.

PR8 virus present in infected lungs was titered by infection of MDCK cells. Flat-bottom 96-well plates were seeded with 1.8×10⁴ MDCK cells per well, and 24 hrs later the serum-containing medium was removed. 30 μl of lung homogenate, either undiluted or diluted from 1×5⁻¹ to 1×5⁻⁷, was inoculated into triplicate wells. After 1 h incubation, 170 μl of infection medium with 4 μg/ml of trypsin was added to each well. Following 48-h incubation at 37° C., the presence or absence of virus was determined by hemagglutination of chicken RBC by supernatant from infected cells. The hemagglutination assay was carried out in V-bottom 96-well plates. Serial 2-fold dilutions of supernatant were mixed with an equal volume of a 0.5% suspension (vol/vol) of chicken erythrocytes (Charles River Laboratories) and incubated on ice for 1 h. Wells containing an adherent, homogeneous layer of erythrocytes were scored as positive. The viral titers were determined by interpolation of the dilution end point that infected 50% of wells by described by S1. Reed, L. J. and H. Muench, “A simple method for estimating fifty percent endpoints,” Am. J. Hyg. 27:493, 1938. TCID₅₀. Assays were performed according to procedures described in Ge, Q., et al., Proceedings of the National Academy of Science 101(23):8676-8681.

Mean Delivery Efficiency

Mean delivery efficiency was determined experimentally. A predetermined amount of powder was weighed into the insuffulator. The weight of the insuffulator was taken before dosing and after dosing. The change in weight from dosing divided by the predetermined total weight was used as the percent delivery efficiency. All values were then averaged.

HPLC Purity

siRNA purity was measured after spray drying by Ion exchange chromatography to determine the percent degradation during spray drying.

Particle Size

The volume median diameter (VMD) was determined on a Rodos/Helos particle size laser diffraction analyzer where one to five milligrams of the dry powder was placed into the inlet on the Helos dry particle size hopper. The particle sizer disperses the dry powder, and a particle size was measured. The experiment was repeated 3 times and an average particle size was taken.

### Example 3

In this example (lot 22-23), the dry powder formulation was siRNA, DPPC, sucrose, and albumin (20:40:20:20 by weight). To prepare this example, an aqueous solution containing 150 mg of siRNA, 150 mg of albumin, and 148 mg of sucrose (total volume 75 ml) was mixed with 175 ml of ethanol containing 299 mg of DPPC. Prior to combining the solutions they were mixed with a magnetic stir bar. After the aqueous solution was added to the organic solution the combined solution was mixed by magnetic stir bar, at room temperature for approximately 6 minutes before the solution was spray dried. Conditions for spray drying were Tᵢₙₗₑₜ=95° C., Tₒᵤₜₗₑₜ=˜55° C., atomization/drying gas flow rate was 600 L/hr.

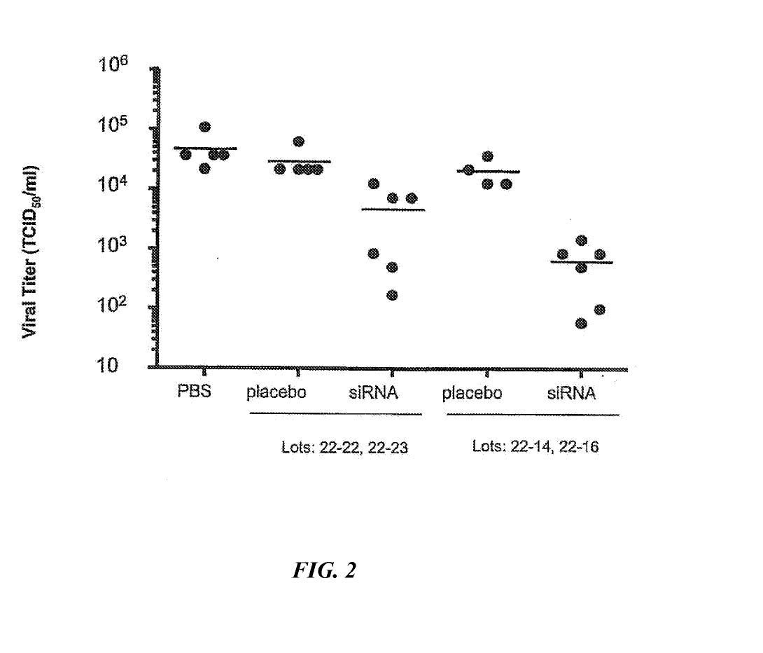

As shown in FIG. 2, and summarized in Table 2 (see below, Example 10), this formulation exhibited an average delivery efficiency of 59.64%. This formulation, targeting the NP protein, inhibited viral titers by 83.9% as compared with a formulation that did not contain the virus targeting siRNA (placebo, lot 22-22).

The VMD of the placebo and active formulation of this example are shown in Table 3 (see below, Example 11).

The purity of the siRNA after spray drying was determined, and the purity of the active formulation of this example was 97.04%, as shown in Table 4 (see below, Example 12).

### Example 4

In this example (lot 22-16), the dry powder formulation was siRNA, DPPC, lactose, and protamine (20:45:30:5 by weight). To prepare this example, an aqueous solution containing 150 mg of siRNA, 43 mg of protamine sulfate, and 223 mg of lactose (total volume 75 ml) was mixed with 175 ml of ethanol containing 332 mg of DPPC. Prior to combining the solutions they were mixed with a magnetic stir bar. After the aqueous solution was added to the organic solution the solution was mixed by magnetic stir bar, at room temperature for approximately 5 minutes before the solution was spray dried. Conditions for spray drying were Tᵢₙₗₑₜ=95° C., Tₒᵤₜₗₑₜ=˜55° C., atomization/drying gas flow rate was 600 L/hr.

As shown in FIG. 2, and summarized in Table 2 (see below, Example 10), this formulation exhibited an average delivery efficiency of 61.62%. This formulation, targeting the NP protein, inhibited viral titers by 96.9% as compared with a formulation that did not contain the virus targeting siRNA (placebo, lot 22-14).

The VMD of the placebo and active formulation of this example are shown in Table 3 (see below, Example 11).

The purity of the siRNA after spray drying was determined, and the purity of the active formulation of this example was 98.10%, as shown in Table 4 (see below, Example 12).

### Example 5

In this example (lot 22-42), the dry powder formulation was siRNA, DPPC, lactose, and arginine (20:45:30:5 by weight). To prepare this example, an aqueous solution containing 150 mg of siRNA, 34 mg of arginine, and 227 mg of lactose (total volume 75 ml) was mixed with 175 ml of ethanol containing 338 mg of DPPC. Prior to combining the solutions they were mixed with a magnetic stir bar. After the aqueous solution was added to the organic solution the solution was mixed by magnetic stir bar, at room temperature for approximately 5 minutes before the solution was spray dried. Conditions for spray drying were Tᵢₙₗₑₜ=95° C., Tₒᵤₜₗₑₜ=˜55° C., atomization/drying gas flow rate was 600 L/hr.

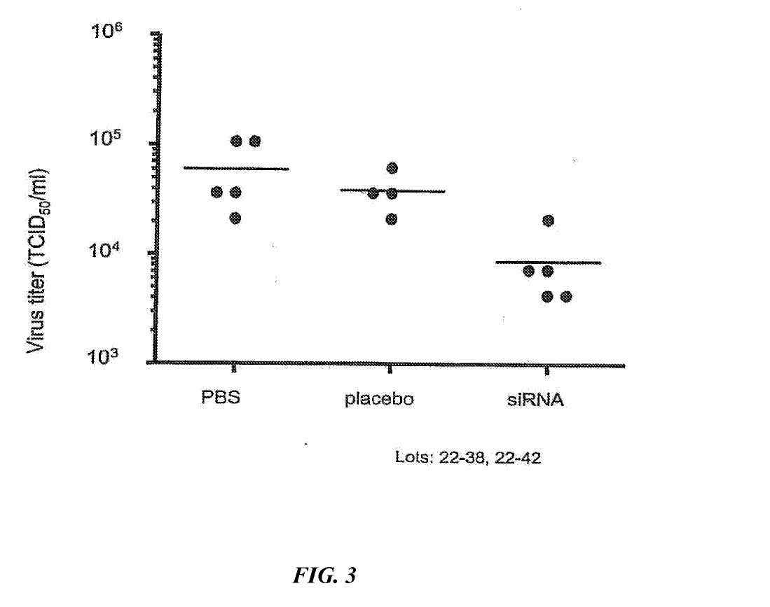

As shown in FIG. 3, and summarized in Table 2 (see below, Example 10), this formulation exhibited an average delivery efficiency of 68.30%. This formulation, targeting the NP protein, inhibited viral titers by 85.5% as compared with a formulation that did not contain the virus targeting siRNA (placebo, lot 22-38).

The VMD of the placebo of this example is shown in Table 3 (see below, Example 11).

The purity of the siRNA after spray drying was determined, and the purity of the active formulation of this example was 99.75%, as shown in Table 4 (see below, Example 12).

### Example 6

In this example (lot no. 22-20), the dry powder formulation was siRNA, DPPC, lactose, and calcium chloride (20:47:30:3 by weight). To prepare this example, an aqueous solution containing 150 mg of siRNA, 23 mg of calcium chloride, and 225 mg of lactose (total volume 75 ml) was mixed with 175 ml of ethanol containing 352 mg of DPPC. Prior to combining the solutions they were mixed with a magnetic stir bar. After the aqueous solution was added to the organic solution the solution was mixed by magnetic stir bar, at room temperature for approximately 6 minutes before the solution was spray dried. Conditions for spray drying were Tᵢₙₗₑₜ=95° C., Tₒᵤₜₗₑₜ=˜55° C., atomization/drying gas flow rate was 600 L/hr.

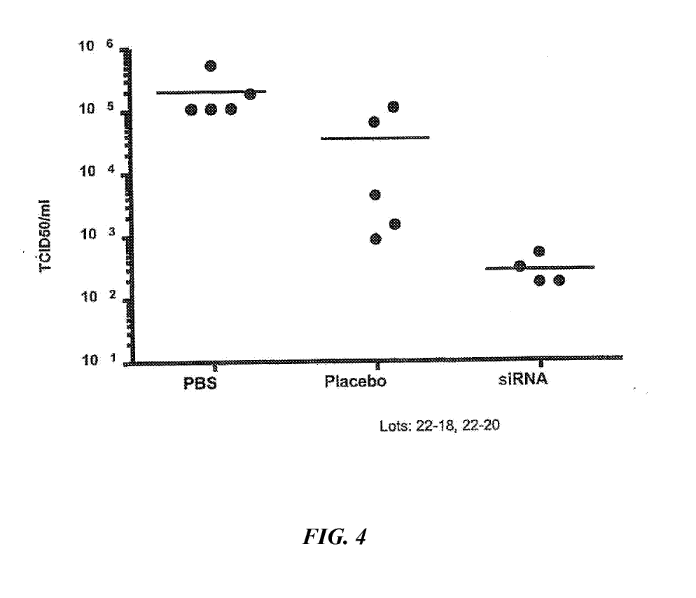

As shown in FIG. 4, and summarized in Table 2 (see below, Example 10), this formulation exhibited an average delivery efficiency of 55.47%. This formulation, targeting the NP protein, inhibited viral titers by 99% as compared with a formulation that did not contain the virus targeting siRNA (placebo, lot 22-18).

The VMD of the placebo and active formulation of this example are shown in Table 3 (see below, Example 11).

The purity of the siRNA after spray drying was determined, and the purity of the active formulation of this example was 99.85%, as shown in Table 4 (see below, Example 12).

### Example 7

In this example (lot 22-67), the dry powder formulation was siRNA, DPPC, leucine, and calcium chloride (20:47:30:3 by weight). To prepare this example, an aqueous solution containing 75 mg of siRNA, 11 mg of calcium chloride, and 113 mg of lactose (total volume 37.5 ml) was mixed with 87.5 ml of ethanol containing 177 mg of DPPC. Prior to combining the solutions they were mixed with a magnetic stir bar. After the aqueous solution was added to the organic solution the solution was mixed by magnetic stir bar, at room temperature for approximately 5 minutes before the solution was spray dried. Conditions for spray drying were Tᵢₙₗₑₜ=95° C., Tₒᵤₜₗₑₜ=˜50° C., atomization/drying gas flow rate was 600 L/hr.

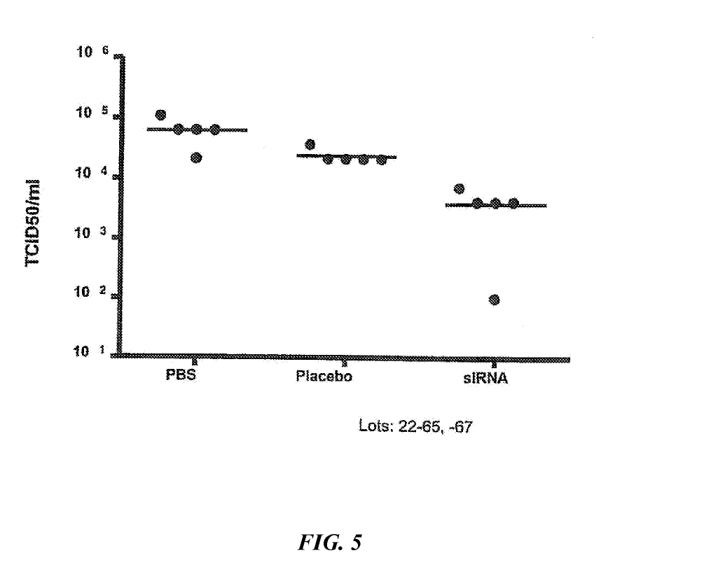

As shown in FIG. 5, and summarized in Table 2 (see below, Example 10), this formulation exhibited an average delivery efficiency of 42.74%. This formulation, targeting the NP protein, inhibited viral titers by 83.7% as compared with a formulation that did not contain the virus targeting siRNA (placebo, lot 22-65).

### Example 8

In this example (lot 22-69), the dry powder formulation was siRNA, DPPC, lactose, and calcium chloride (20:47:30:3 by weight). To prepare these lots, an aqueous solution containing 75 mg of siRNA, and 13 mg of calcium chloride, (total volume 11.25 ml) was mixed with 26.25 ml ethanol. The solution was incubated overnight at −20° C. The next day 113 mg of lactose was dissolved in 26.25 ml of nuclease free water and 175 mg of DPPC was dissolved in ethanol. The aqueous phase was then added to the organic phase. The precipitated solution was added after the solutions were combined. Afterward, the solutions were combined and mixed by magnetic stir bar, at room temperature for approximately 5 minutes before being spray dried. Conditions for spray drying were Tᵢₙₗₑₜ=95° C., Tₒᵤₜₗₑₜ=˜50° C., atomization/drying gas flow rate was 600 L/hr.

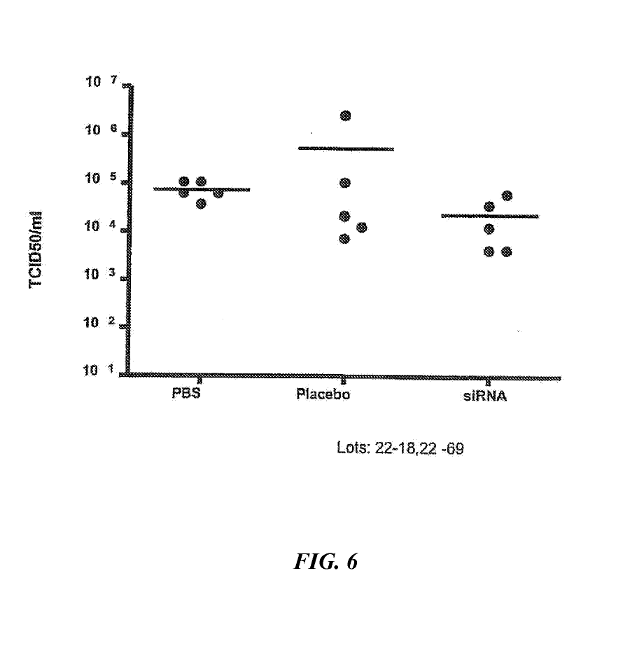

As shown in FIG. 6, and summarized in Table 2 (see below, Example 10), this formulation exhibited an average delivery efficiency of 37.76%. This formulation, targeting the NP protein, inhibited viral titers by 95.74% as compared with a formulation that did not contain the virus targeting siRNA (placebo, lot 22-18).

The VMD of the placebo and active formulation of this example are shown in Table 3 (see below, Example 11).

### Example 9

In this example (22-73), the dry powder formulation was siRNA, DPPC, lactose, and calcium chloride (20:45:30:5 by weight). To prepare these lots, an aqueous solution containing 75 mg of siRNA, 11 mg of calcium chloride, and 113 mg of lactose (total volume 37.5 ml) was mixed with 87.5 ml of ethanol containing 176 mg of DPPC. Prior to combining the solutions they were mixed with a magnetic stir bar. After the aqueous solution was added to the organic solution the solution was mixed by magnetic stir bar, at room temperature for approximately 2 minutes before the solution was spray dried. Conditions for spray drying were Tᵢₙₗₑₜ=95° C., Tₒᵤₜₗₑₜ=˜50° C., atomization/drying gas flow rate was 600 L/hr.

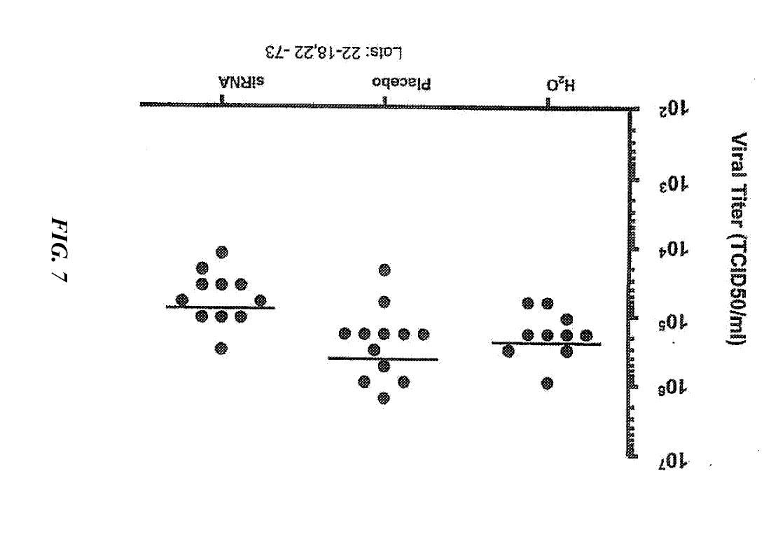

As shown in FIG. 7, and summarized in Table 2 (see below, Example 10), this formulation exhibited an average delivery efficiency of 24.88%. This formulation, targeting the NP protein, inhibited viral titers by 81.20% as compared with a formulation that did not contain the virus targeting siRNA (placebo, lot 22-18).

### Example 10

The efficiency and effectiveness of the dry powder formulations of Examples 3-9 are summarized in Table 2.

TABLE 2
Summary of Efficiency and Effectiveness of Example Dry Powder Formulations

| | | | | | Average | |
|---|---|---|---|---|---|---|
| Ex. | | | | | Delivery | Percent |
| No. | Lot | Composition | Ratio | Dose | Efficiency | Silencing |
| 3 | 22-22 | DPPC:sucrose:albumin | 40:20:20 | 1 mg | 43.16% | |
| 3 | 22-23 | DPPC:sucrose:albumin:siRNA | 40:20:20:20 | 1 mg | 59.64% | 83.90% |
| 4 | 22-14 | DPPC:lactose:protamine | 40:30:5 | 1 mg | 85.94% | |
| 4 | 22-16 | DPPC:lactose:protamine:siRNA | 40:30:5:20 | 1 mg | 61.62% | 96.90% |
| 5 | 22-38 | DPPC:lactose:arginine | 45:30:5 | 1 mg | 74.79% | |
| 5 | 22-42 | DPPC:lactose:arginine:siRNA | 45:30:5:20 | 1 mg | 68.3% | 85.50% |
| 6 | 22-18 | DPPC:lactose:CaCl2 | 47:30:3 | 1 mg | 60.89% | |
| 6 | 22-20 | DPPC:lactose:CaCl2:siRNA | 47:30:3:20 | 1 mg | 55.47% | 99% |
| 7 | 22-65 | DPPC:leucine:calcium chloride | 47:30:3 | 1.5 mg | 63.15% | |
| 7 | 22-67 | DPPC:leucine:CaCl2:siRNA | 47:30:3:20 | 2 mg | 42.74% | 83.7% |
| 8 | 22-18 | DPPC:lactose:CaCl2 | 47:30:3 | 1.5 mg | 65.79% | |
| 8 | 22-69 | DPPC:lactose:CaCl2:siRNA | 47:30:3:20 | 2 mg | 37.76% | 95.74% |
| 9 | 22-18 | DPPC:lactose:CaCl2 | 47:30:3 | 1 mg | 58.71% | |
| 9 | 22-73 | DPPC:lactose:CaCl2:siRNA | 47:30:3:20 | 2 mg | 24.88% | 81.2% |

### Example 11

The Volume Median Diameter of the dry powder formulations of Examples 3, 4, 6, and 8 and the placebo formulations of Examples 3, 4, 5, 6, and 8 are summarized in Table 3.

TABLE 3
Volume Median Diameter for Example Dry Powder Formulations

| Lot Number | VMD Average (N = 3) | VMD Standard Deviation |
|---|---|---|
| 22-14 | 1.70 | 0.21 |
| 22-16 | 1.49 | 0.02 |
| 22-18 | 1.24 | 0.01 |
| 22-20 | 1.54 | 0.02 |
| 22-22 | 2.13 | 0.53 |
| 22-23 | 1.34 | 0.01 |
| 22-38 | 1.32 | 0.03 |
| 22-69 | 1.69 | 0.24 |

### Example 12

The purity of the dry powder active formulations of Examples 3-6 are summarized in Table 4.

TABLE 4
Purity of siRNA Upon Formulation

| | % Purity |
|---|---|
| Lot Number | (compared to purity of siRNA starting material) |
| 22-16 | 98.10% |
| 22-20 | 99.85% |
| 22-23 | 97.04% |
| 22-42 | 99.75% |

### Example 13

NP Transcript Sequence (+sense)

Influenza A strain PR8

1565 bases, 5′->3′

(SEQ. ID NO.: 17)

AGCAAAAGCAGGGTAGATAATCACTCACTGAGTGACATCAAAATCATGGC
GTCCCAAGGCACCAAACGGTCTTACGAACAGATGGAGACTGATGGAGAAC
GCCAGAATGCCACTGAAATCAGAGCATCCGTCGGAAAAATGATTGGTGGA
ATTGGACGATTCTACATCCAAATGTGCACCGAACTCAAACTCAGTGATTA
TGAGGGACGGTTGATCCAAAACAGCTTAACAATAGAGAGAATGGTGCTCT
CTGCTTTTGACGAAAGGAGAAATAAATACCTGGAAGAACATCCCAGTGCG
GGGAAAGATCCTAAGAAAACTGGAGGACCTATATACAGGAGAGTAAACGG
AAAGTGGATGAGAGAACTCATCCTTTATGACAAAGAAGAAATAAGGCGAA
TCTGGCGCCAAGCTAATAATGGTGACGATGCAACGGCTGGTCTGACTCAC
ATGATGATCTGGCATTCCAATTTGAATGATGCAACTTATCAGAGGACAAG
AGCTCTTGTTCGCACCGGAATGGATCCCAGGATGTGCTCTCTGATGCAAG
GTTCAACTCTCCCTAGGAGGTCTGGAGCCGCAGGTGCTGCAGTCAAAGGA
GTTGGAACAATGGTGATGGAATTGGTCAGGATGATCAAACGTGGGATCAA
TGATCGGAACTTCTGGAGGGGTGAGAATGGACGAAAAACAAGAATTGCTT
ATGAAAGAATGTGCAACATTCTCAAAGGGAAATTTCAAACTGCTGCACAA
AAAGCAATGATGGATCAAGTGAGAGAGAGCCGGAACCCAGGGAATGCTGA
GTTCGAAGATCTCACTTTTCTAGCACGGTCTGCACTCATATTGAGAGGGT
CGGTTGCTCACAAGTCCTGCCTGCCTGCCTGTGTGTATGGACCTGCCGTA
GCCAGTGGGTACGACTTTGAAAGAGAGGGATACTCTCTAGTCGGAATAGA
CCCTTTCAGACTGCTTCAAAACAGCCAAGTGTACAGCCTAATCAGACCAA
ATGAGAATCCAGCACACAAGAGTCAACTGGTGTGGATGGCATGCCATTCT
GCCGCATTTGAAGATCTAAGAGTATTAAGCTTCATCAAAGGGACGAAGGT
GCTCCCAAGAGGGAAGCTTTCCACTAGAGGAGTTCAAATTGCTTCCAATG
AAAATATGGAGACTATGGAATCAAGTACACTTGAACTGAGAAGCAGGTAC
TGGGCCATAAGGACCAGAAGTGGAGGAAACACCAATCAACAGAGGGCATC
TGCGGGCCAAATCAGCATACAACCTACGTTCTCAGTACAGAGAAATCTCC
CTTTTGACAGAACAACCATTATGGCAGCATTCAATGGGAATACAGAGGGA
AGAACATCTGACATGAGGACCGAAATCATAAGGATGATGGAAAGTGCAAG
ACCAGAAGATGTGTCTTTCCAGGGGCGGGGAGTCTTCGAGCTCTCGGACG
AAAAGGCAGCGAGCCCGATCGTGCCTTCCTTTGACATGAGTAATGAAGGA
TCTTATTTCTTCGGAGACAATGCAGAGGAGTACGACAATTAAAGAAAAAT
ACCCTTGTTTCTACT.

What is claimed is:

1. A dry powder formulation for mucosal, intranasal, inhalation or pulmonary delivery comprising one or more siRNAs or dicer-active precursors thereof targeted to a transcript involved in infection by, or replication or production of an influenza virus.

2. The formulation of claim 1, wherein the siRNAs are targeted to a plurality of influenza virus strains.

3. The formulation of claim 1, wherein the siRNAs are targeted to two or more regions of the same influenza virus transcript.

4. The formulation of claim 1, the siRNAs comprising a core duplex region having sense and antisense strand portions, the sequence of the antisense strand portion of the core duplex region comprising at least 10 consecutive nucleotides as set forth in nucleotides 1 through 19 of the sequence presented in any one of SEQ ID NOS: 2, 4, 6, 8, 10, 12, 14, and 16, with the proviso that either one or two nucleotides among the 10 consecutive nucleotides may differ from the sequence presented in any one of SEQ ID NOS: 2, 4, 6, 8, 10, 12, 14, and 16.

5. The formulation of claim 1, the siRNAs comprising a core duplex region having sense and antisense strand portions, the sequence of the antisense strand portion of the core duplex region comprising nucleotides 1 through 19 of the sequence presented in any one of SEQ ID NOS: 2, 4, 6, 8, 10, 12, 14, and 16, the siRNA.

6. The formulation of claim 1, wherein the siRNA is G1498.

7. The formulation of claim 1, the siRNAs having at least one 5-methyluridine-modified nucleotide.

8. A dry powder aerosolizable formulation for mucosal, intranasal, inhalation or pulmonary delivery comprising one or more siRNAs selected from the group of G3789, G3807, G3817, G6124, G6129, G8282, G8286, and G1498, along with DPPC, and a carrier.

9. The formulation of claim 8, wherein the siRNA is G1498 and the carrier is lactose.

10. The formulation of claim 8, further comprising a buffer agent.

11. The formulation of claim 10, wherein the buffer agent is calcium chloride.

12. The formulation of claim 8, further comprising a protein.

13. The formulation of claim 12, wherein the protein is albumin.

14. The formulation of claim 8, further comprising an amino acid.

15. The formulation of claim 14, wherein the amino acid is arginine.

16. The formulation of claim 8, the powder comprising particles having an MMD of from about 1.5 to 5.5 μm.

17. The formulation of claim 8, the powder comprising particles having an MMAD of from about 1 to 6 μm.

18. The formulation of claim 8, wherein the fine particle fraction (FPF) of the powder is from about 20 to about 70%.

19. The formulation of claim 8, wherein the purity of the siRNA is greater than about 90% by weight upon storage of the under ambient conditions for a period of 18 months.

20. A method of treating or preventing influenza in an animal comprising administering a therapeutically effective amount of the formulation of claims 1 or 8 to the animal.

21. The method of claim 19, wherein the siRNA is G1498.

22. A method of inhibiting the replication or production of an influenza virus in an animal comprising administering a therapeutically effective amount of the formulation of claims 1 or 8 to the animal.
